(19) United States
(12) Patent Application Publication
(10) Pub. No.: US 2026/0014276 A1
(43) Pub. Date: Jan. 15, 2026

(54) AN OPTOGENETIC GENE THERAPY FOR TREATING BLINDNESS

(71) Applicant: Bionic Sight, LLC, New York, NY (US)

(72) Inventor: Sheila Nirenberg, New York, NY (US)

(73) Assignee: Cornell University, Ithaca, NY (US)

(21) Appl. No.: 18/992,942

(22) PCT Filed: Jul. 6, 2023
(86) PCT No.: PCT/US2023/069705

§ 371 Date: Jan. 9, 2025

Related U.S. Application Data
(60) Provisional application No. 63/359,822, filed on Jul. 9, 2022.

(51) Int. Cl. A61K 48/00; A61K 9/00; A61K 31/522; A61K 31/573; A61N 5/06; A61P 27/02; C12N 15/86

(52) U.S. Cl. CPC A61K 48/005; A61K 9/0048; A61K 31/522; A61K 31/573; A61K 48/0075; A61K 48/0083; A61N 5/062; A61N 5/0622; A61P 27/02; C12N 15/86; C12N 2750/14143

(57) ABSTRACT

Provided herein is an AAV2 vector encoding SEQ ID NO: 4 as the optogenetic protein, and methods of use for e.g., retinitis pigmentosa.

### CROSS REFERENCE TO RELATED APPLICATIONS

This application claims the benefit of U.S. Provisional Application No. 63/359,822 filed Jul. 9, 2022, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Jun. 26, 2023, is named BIS-001WO_SL.xml and is 20,730 bytes in size.

### BACKGROUND

Age-related macular degeneration (AMD) and retinitis pigmentosa (RP), together, affect 2 million people in the U.S. and 25 million people worldwide. In both retinal degenerative diseases, it is the input of the retina that degenerates: cones degenerate in AMD and rods in RP. More particularly, RP is a large group of inherited retinal disorders in which progressive degeneration of photoreceptors or retinal pigment epithelium leads to vision loss. The clinical manifestations of affected individuals present first as night blindness, followed by reduction of peripheral vision and, eventually, loss of central vision.

Current treatments for patients with retinal degenerative diseases are limited. There is some evidence to suggest that vitamin A and fish oil supplements may slow vision loss in some patients with early disease, but they are not able to reverse the disease. For a subset of patients whose retinal degeneration is caused a mutation in the RPE65 gene, targeted gene therapy is now possible and is currently being used to treat patients. However, because RP, for example, is a genetically heterogenous disease, with more than 100 different genes or loci that lead to the common endpoint of vision loss, gene therapy is not necessarily applicable to the majority of patients.

New gene therapies that employ optogenetics, however, are opening new therapeutic options for patients with RP. With optogenetics, it is possible to treat the disease in a way that is independent of the underlying gene defect, allowing a much broader range of patients to obtain benefit. While highly promising as a concept, these type approaches may have limitations as a therapy due to, for example, requiring very bright light to activate the protein and/or phototoxic effects due to short excitation wavelength.

Therefore there is a significant unmet need for treatment modalities (e.g., for RP) in human patients that can improve eyesight, and, for example, provide patients with enhanced ability to detect light, shapes, movement, and/or color.

### SUMMARY

The disclosure herein at least in part provides compositions and methods for treating a retinal degenerative disorder, and provides, for example an adeno-associated viral (AAV) 2 (AAV2) vector (e.g., a sequence having 95%, 96%, 97%, 98%, 99% identity to SEQ ID NO: 1) having a gene that expresses an optogenetic fusion protein (e.g., a nucleic acid molecule with the nucleic acid sequence of SEQ ID NO: 2 (or e.g., a sequence having 95%, 96%, 97%, 98% or 99% identity to SEQ ID NO: 2) or a nucleic acid encoding a protein having 98% or 99% identity to the amino acid sequence of SEQ ID NO: 3. Such contemplated AAV vectors may be administered intravitreally to a patient with, for example, an initial dose (e.g., administered to the retinal surface of the patient's eye).

For example, disclosed methods of delivering contemplated optogenetic proteins to the human eye provide substantially more light sensitivity (more than 100-fold) than expected from measurements of light sensitivity produced by the protein in cultured neurons. Patients with complete or near complete blindness, who received the treatment with e.g., SEQ ID. NO 1 were able to detect light at much lower light levels, including daylight and television light levels, than expected from published reports with the protein.

Also provided herein are methods that include administering an effective amount of a vector dose (e.g., a contemplated vector herein, e.g., SEQ ID NO: 9) of about 1×10¹¹ to about 1×10¹³ vector genomes (vg)/eye. Surprisingly, for example, upon administration, the dose response of about 1.2×10¹² vg/eye or more produced an unexpected supralinear improvement in light sensitivity e.g., the amplitude of electroretinogram (ERG) responses in mice shows an approximately linear dose-response with lower doses, but a supralinear increase in amplitude at these higher doses.

Contemplated methods may further include administering to the patient a light delivery device that sends light pulses to the optogenetic protein in the retina's neural code, causing the retina's output cells, the ganglion cells, to fire in patterns that mimic those of the normal retina. Surprisingly, administration of both a contemplated vector and neurally coded stimulation, provides patients (e.g., those who received the treatment, to detect motion, the direction of the motion, and, for some patients, the ability to recognize objects).

For example, or patients who are moderately blind, upon administration of a contemplated vector e.g., SEQ ID NO: 9 the interaction of the vector with the patient's residual retinal circuitry, provides an unexpected boost to his/her ability to detect shapes, count fingers, and recognize objects. Also surprisingly, e.g., for patients who are moderately blind, upon administration of a contemplated vector, as provided herein, the interaction of the patient's cone system allows for recognition of colors.

DNA polynucleotide encoding an optogenetic fusion protein or an RNA equivalent thereof: wherein the optogenetic fusion protein includes a light-activated ion channel protein fused to a reporter protein, wherein the light-activated ion channel protein has the amino acid sequence of SEQ ID NO: 5, wherein the reporter protein is fused to the 3′ end of the optogenetic protein, and wherein the polynucleotide is operably linked to a CAG promoter having the nucleic acid sequence of SEQ ID NO: 6 and a WPRE enhancer having the nucleic acid sequence of SEQ ID NO: 7.

This disclosure is based, at least in part, on the surprising discovery that a vector encoding a light-sensitive protein (e.g., a nucleic acid molecule having the nucleic acid sequence of SEQ ID NO: 4 or a nucleic acid encoding a protein having the amino acid sequence of SEQ ID NO: 5) was efficacious in humans over a broad range of vector doses and stimulating light intensities. Further, contemplated vectors upon administration to human patients are well tolerated. In some embodiments, the disclosure provides a combination therapy, including optogenetic gene therapy (e.g., with a vector encoding a light-sensitive protein (e.g., a nucleic acid molecule having the nucleic acid sequence of SEQ ID NO: 4 or a nucleic acid encoding a protein having the amino acid sequence of SEQ ID NO: 5) and neurally-coded stimulation. The combination therapy described herein has the surprising advantage of providing unprecedented vision restoration to patients with a retinal degenerative disorder, such as patients with advanced stage blindness due to retinitis pigmentosa. The compositions and methods described herein can, thus, be used to restore vision to patients with retinal degenerative diseases.

### BRIEF DESCRIPTION OF THE DRAWINGS

FIGS. 1A-1B are a set of graphs depicting that administration of Compound A, an adeno-associated virus 2 (AAV2) whose genome is the nucleic acid sequence of SEQ. ID NO: 1 and whose expression cassette is the nucleic acid sequence of SEQ ID NO: 9, produces robust electroretinograms (ERG) responses in Pde6brd1 (rd1) mice. FIG. 1A is a set of graphs depicting raw ERG responses (mean±SEM) from an untreated eye (top, red) and an Compound A treated eye (bottom, blue) from the same animal. Horizontal black lines on each plot indicate the locations of the stimulus artifacts, e.g., small upward and downward electrical transients at stimulus onset and offset. To the right of the bottom panel are a set of traces from separate non-overlapping epochs in the ERG, indicating consistency over the entire acquisition period. FIG. 1B is a set of graphs of ERG responses for all animals. For each eye in each animal, the response was quantified by the size of the photopic negative response (PhNR)-like wave (e.g., the ERG component that corresponds to the ganglion cell response), measured as the difference between the voltage at time zero and the average voltage during the last half of the stimulation period, just prior to the stimulus artifact. A PhNR-like wave was observed in 6 of the 7 animals examined. The size of the wave was significant both at a group level (p<0.001, unpaired t-test), and at the level of the individual animals (p<0.004, paired t-test, comparing, for each animal, its treated eye with its untreated counterpart, shown on the right). Some variance in the responses is expected due to injection variance in a small target (mouse eye). Outcomes were measured 10 weeks after vector injection. Light stimulation was 0.06 mW/mm2, 505 nm.

FIGS. 2A-2B are a set of graphs depicting the assessment of ERG responses in rd1 mice over a 20-fold dose range. FIG. 2A is a graph depicting ERG responses to light stimulation from animals that were treated with Compound A. Five groups are shown: untreated eyes (n=7), eyes treated with a dose of 5×10⁷ vg/eye (n=7), eyes treated with a dose of 1×10⁸ vg/eye (n=3), eyes treated with a dose of 5×10⁸ vg/eye (n=12), and eyes treated with a dose of 1×10⁹ vg/eye (n=11). The mean response amplitude for each dose group was statistically significantly different from that of the control (p<0.01), and, as expected, response amplitude increased with increasing dose. FIG. 2B is a set of graphs depicting raw ERG responses (mean±SEM) from each of the five groups. The response for each eye was quantified by the size of the PhNR-like wave. All injections were performed 10-15 weeks prior to recording. Light stimulation was 0.06 mW/mm², 505 nm.

FIG. 3 is a graph depicting the assessment of ERG responses in Compound A-treated rd1 mice to lower light levels. For animals treated with the two highest doses, 1×10⁹ vg/eye and 5×10⁸ vg/eye, light levels could be substantially reduced from the level used on FIGS. 2A-2B and 3 (0.06 mW/mm²) and still produce ERG responses that were well above baseline (see untreated eyes in FIG. 2A-2B) (p<0.01). All injections were performed 10-15 weeks prior to recording.

FIG. 4 is a graph depicting that there is no loss of retinal ganglion cells in Compound A-treated retinas compared to untreated retinas. Mean density of Brn3a-positive cells from control retinas, low dose Compound A-treated retinas, and high dose Compound A-treated retinas. Data are plotted as the number of ganglion cells per linear mm of retina (mean±SEM). No statistically significant difference was observed in retinal ganglion cell counts between the low dose group and the control group (p>0.1, Student's t-test) or between the high dose group and the control group (p>0.5, Student's t-test). The low dose group contained 10 eyes, the high dose group contained 9 eyes, and the control group contained 3 vehicle-treated eyes and 2 untreated eyes.

FIGS. 5A-5B are a set of graphs depicting that there is no loss of Compound A-expressing ganglion cells and photoreceptors in retinas treated with both Compound A and light. FIG. 5A is a graph showing the comparison of the densities (mean±SEM) of Compound A-expressing cells in the Compound A-alone group and the Compound A-plus-light-treated group; no statistically significant difference between the two groups was observed (p>0.7, Student's t-test). FIG. 5B is a graph showing the comparison of the densities (mean±SEM) of cones in the untreated retinas with those in the Compound A-plus-light-treated retinas; no statistically significant difference was observed (p>0.4, Student's 1-test).

FIGS. 6A-6B are a set of graphs depicting that there is no statistically significant drop in the amplitude of ERG components between treated and untreated eyes in nonhuman primates. FIG. 6A is a graph depicting a representative ERG response to a flash stimulus showing the 3 main ERG components: the a-wave, the b-wave, and the PhNR. FIG. 6B is a set of graphs depicting the mean maximum upstroke velocity (Vₘₐₓ) values for the three ERG components in the untreated and treated groups. No statistically significant reduction in Vₘₐₓ was observed for any of the three components as a result of the treatment (p>0.2, for all waves, comparing the Vₘₐₓ values in the treated group with those in the untreated group). The ERGs for the treated animals were performed 7 months after vector injection.

FIGS. 7A-7B are a set of graphs depicting that the distribution of ganglion cell receptive field sizes and firing rates from Compound A-expressing retinas was not statistically significantly different from those of untreated retinas. FIG. 7A is a histogram of receptive field sizes from untreated retina (top) and SEQ ID NO: 1 treated retina (bottom), using a comparable retinal eccentricity (between 3 and 12 mm from central retina) (p>0.2, Kolmogorov-Smirnov test). FIG. 7B is a histogram of ganglion cell firing rates from untreated retinas (top) and Compound A-expressing retinas (bottom); the two distributions were not statistically significantly different (p>0.2, Kolmogorov-Smirnov test). Firing rates were measured in response to movies of natural scenes, including trees, landscapes, and people walking. All eyes were injected intravitreally with a Compound A vector 3-6 months before eye removal for electrophysiological recording.

FIG. 8 is a set of graphs depicting the results from individual human patients in a 2-alternative forced choice task to test light sensitivity. Said patients were intravitreally administrated an initial vector dose of at least 1×10¹¹ vg/eye of Compound A, followed by paradigm testing.

FIG. 9 is a set of graphs depicting the results from individual human patients taken over time (e.g., at months 3, 6, 9, and/or 12) in a 2-alternative forced choice task to test light sensitivity. Said patients were intravitreally administrated an initial vector dose of an effective amount (e.g., at least about 1×10¹¹ vg/eye) of Compound A, followed by paradigm testing.

FIG. 10 is a set of graphs depicting the results from individual human patients in four paradigms, including paradigms to measure the ability to detect motion (“motion”), the ability to detect the direction of motion (“direction”), the ability to distinguish live actions (e.g., arms moving up vs. down or e.g., an arm moving vs. a hand moving; “live”), and the ability to distinguish objects (e.g., an apple vs. other fruits or vegetables or e.g., the suits on playing cards; “object”), respectively. Said patients were intravitreally administrated an initial vector dose of an effective amount (e.g., at least about 1×10¹¹ vg/eye) of Compound A, followed by paradigm testing.

FIG. 11 is a set of graphs depicting the results from individual human patients as a function of time in the four paradigms, described in FIG. 10, including motion, direction, live, and object, respectively. Said patients were intravitreally administrated an initial vector dose of an effective amount (e.g., at least about 1×10¹¹ vg/eye) of Compound A, followed by paradigm testing.

FIGS. 12A-12B are a set of graphs depicting the results of an individual human patient having a baseline visual acuity of <20/200 in counting fingers (FIG. 12A) as well as motion/object recognition (FIG. 12B) tests, respectively. Said patients were intravitreally administrated an initial vector dose of an effective amount of Compound A (e.g., at least about 1×10¹¹ vg/eye), followed by paradigm testing.

FIGS. 13A-13B are a set of graphs depicting the results of a second individual human patient having a baseline visual acuity of <20/200 in counting fingers (FIG. 13A) and motion/object recognition (FIG. 13B) tests, respectively. Said patients were intravitreally administrated an initial vector dose (e.g., at least about 1×10¹¹ vg/eye) of Compound A, followed by paradigm testing.

FIG. 14 is a set of graphs depicting the results of two individual human patients, respectively, having a baseline visual acuity of <20/400 in a color identification test.

FIG. 15 is a graph showing the amplitude of ERG response in mice administered varying doses of Compound A. Doses include, from left to right, untreated, 5×10⁷ vg/eye, 1×10⁸ vg/eye (equivalent to human “Dose 1, 1×10¹¹ vg/eye”), 5×10⁸ vg/eye (approximately equivalent to human “Dose 3, 6×10¹¹ vg/eye”), and 1×10⁹ vg/eye (equivalent to human “Dose 4, 1×10¹² vg/eye”).

FIG. 16 is a depiction of the nucleic acid sequence of Compound A (SEQ ID NO: 1).

### DETAILED DESCRIPTION

### Optogenetic Protein

Compounds of the disclosure include a gene that expresses an optogenetic protein. Optogenetic proteins, in many cases, are light-gated ion channels or pumps that absorb light at specific wavelengths. Upon activation by light, these channels and pumps respond by opening or closing, which allows the flow of ions into or out of the cell in which the protein is respectively expressed in. For example, provided herein is an optogenetic protein that comprises a light-sensitive channel from e.g., Stigeoclonium helveticum, such as an optogenetic protein encoded by a nucleic acid with the nucleic acid sequence of SEQ ID NO: 4, below:

ATGGAAACAGCCGCCACAATGACCCACGCCTTTATCTCAGCCGTG
CCTAGCGCCGAAGCCACAATTAGAGGCCTGCTGAGCGCCGCAGCA
GTGGTGACACCAGCAGCAGACGCTCACGGAGAAACCTCTAACGCC
ACAACAGCCGGAGCCGATCACGGTTGCTTCCCCCACATCAACCAC
GGAACCGAGCTGCAGCACAAGATCGCAGTGGGACTCCAGTGGTTC
ACCGTGATCGTGGCTATCGTGCAGCTCATCTTCTACGGTTGGCAC
AGCTTCAAGGCCACAACCGGCTGGGAGGAGGTCTACGTCTGCGTG
ATCGAGCTCGTCAAGTGCTTCATCGAGCTGTTCCACGAGGTCGAC
AGCCCAGCCACAGTGTACCAGACCAACGGAGGAGCCGTGATTTGG
CTGCGGTACAGCATGTGGCTCCTGACTTGCCCCGTGATCCTGATC
CACCTGAGCAACCTGACCGGACTGCACGAAGAGTACAGCAAGCGG
ACCATGACCATCCTGGTGACCGACATCGGCAACATCGTGTGGGGG
ATCACAGCCGCCTTTACAAAGGGCCCCCTGAAGATCCTGTTCTTC
ATGATCGGCCTGTTCTACGGCGTGACTTGCTTCTTCCAGATCGCC
AAGGTGTATATCGAGAGCTACCACACCCTGCCCAAAGGCGTCTGC
CGGAAGATTTGCAAGATCATGGCCTACGTCTTCTTCTGCTCTTGG
CTGATGTTCCCCGTGATGTTCATCGCCGGACACGAGGGACTGGGC
CTGATCACACCTTACACCAGCGGAATCGGCCACCTGATCCTGGAT
CTGATCAGCAAGAACACTTGGGGCTTCCTGGGCCACCACCTGAGA
GTGAAGATCCACGAGCACATCCTGATCCACGGCGACATCCGGAAG
ACAACCACCATCAACGTGGCCGGCGAGAACATGGAGATCGAGACC
TTCGTCGACGAGGAGGAGGAGGGAGGAGTG.

In some embodiments, a contemplated optogenetic protein is a protein having the amino acid sequence of SEQ ID NO: 5, below:

METAATMTHAFISAVPSAEATIRGLLSAAAVVTPAADAHGETSNA
TTAGADHGCFPHINHGTELQHKIAVGLQWFTVIVAIVQLIFYGWH
SFKATTGWEEVYVCVIELVKCFIELFHEVDSPATVYQTNGGAVIW
LRYSMWLLTCPVILIHLSNLTGLHEEYSKRTMTILVTDIGNIVWG
ITAAFTKGPLKILFFMIGLFYGVTCFFQIAKVYIESYHTLPKGVC
RKICKIMAYVFFCSWLMFPVMFIAGHEGLGLITPYTSGIGHLILD
LISKNTWGFLGHHLRVKIHEHILIHGDIRKTTTINVAGENMEIET
FVDEEEEGGV.

In some embodiments, a contemplated optogenetic protein is fused to a reporter protein.

A reporter protein may include, for example, be a fluorescent protein, a luciferase, beta-galactosidase, alkaline phosphatase, beta-lactamase, a protein or enzyme which confers resistance to cytotoxic substances or to minimal medium, a cytotoxic or pro-apoptotic protein, or a protein which modifies the growth or morphology of the cell in which they are expressed. For example, in some embodiments, the reporter protein fused to an optogenetic protein is a fluorescent protein, e.g., luciferase and/or the reporter protein may be fused to an optogenetic protein that includes one of: beta-galactosidase, alkaline phosphatase or beta-lactamase. In some embodiments, the reporter protein fused to an optogenetic protein is a protein or enzyme which confers resistance to cytotoxic substances or to minimal medium, e.g., a cytotoxic or pro-apoptotic protein and/or protein which modifies the growth or morphology of the cell in which they are expressed.

In some embodiments, the reporter protein fused to an optogenetic protein is a fluorescent protein, e.g., a reporter protein is fused to the 3′ end of the optogenetic protein.

Fluorescent proteins of the disclosure may be any suitable fluorescent protein, such as a green fluorescent protein a blue fluorescent protein, a cyan fluorescent protein, a yellow fluorescent protein, an orange fluorescent protein, or a red fluorescent protein. For example, the reporter protein is a green fluorescent protein.

Exemplary fluorescent proteins maybe selected from green fluorescent protein (GFP) (e.g., with an excitation maximum 395/475 nm, emission maximum 509 nm and relative brightness (e.g., % of EGFP 48%), as well as green fluorescent proteins such as EFTP, Emerald, superfolder GFP, Azami Green, mWasabi, TagGFP, TurboGFP, AcGFP, ZsGreen, or T-Sapphire, blue fluorescent proteins such as EBFP, EBFP2, Azurite, mTagBFP, cyan fluorescent proteins such as ECFP, mECFP, cerulean, m Turqoise, CyPet, AmCyan1, Midori-Ishi Cyan, TagCFP, mTFP1 (Teal), yellow fluorescent proteins such as EYFP, Topaz, Venus, mCitrine, YPet, TagYFP, PhiYFP, ZsYellow1 or mBanana; Orange Fluorescent Proteins such as Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine; Red Fluorescent Proteins such as mRuby, mApple, mStrawberry, AsRed2, mRFP1, JRed, mCherry, HcRed1, mRaspberry, dKeima-Tandem, HcRed-Tandem, mPlum, or AQ143.

In some embodiments, the fluorescent is a green fluorescent protein. For example, in some embodiments, the green fluorescent protein is GFP. In some embodiments, the green fluorescent protein is EGFP. In some embodiments, the green fluorescent protein is Emerald. In some embodiments, the green fluorescent protein is superfolder GFP. In some embodiments, the green fluorescent protein is Azami Green. In some embodiments, the green fluorescent protein is mWasabi. In some embodiments, the green fluorescent protein is TagGFP. In some embodiments, the green fluorescent protein is TurboGFP. In some embodiments, the green fluorescent protein is AcGFP. In some embodiments, the green fluorescent protein is ZsGreen. In some embodiments, the green fluorescent protein is T-Sapphire.

Contemplated optogenetic fusion proteins may be encoded by a nucleic acid sequence that is at least 94% identical to the nucleic acid molecule of SEQ ID NO: 2. In some embodiments, the optogenetic fusion protein is encoded by nucleic acid sequence that is at least 95%, 96%, 97%, 98%, or 99% identical to the nucleic acid molecule of SEQ ID NO: 2.

For example, the optogenetic fusion protein may be encoded by a nucleic acid molecule having the nucleic acid sequence of SEQ ID NO: 2, below:

ATGGAAACAGCCGCCACAATGACCCACGCCTTTATCTCAGCCGTG
CCTAGCGCCGAAGCCACAATTAGAGGCCTGCTGAGCGCCGCAGCA
GTGGTGACACCAGCAGCAGACGCTCACGGAGAAACCTCTAACGCC
ACAACAGCCGGAGCCGATCACGGTTGCTTCCCCCACATCAACCAC
GGAACCGAGCTGCAGCACAAGATCGCAGTGGGACTCCAGTGGTTC
ACCGTGATCGTGGCTATCGTGCAGCTCATCTTCTACGGTTGGCAC
AGCTTCAAGGCCACAACCGGCTGGGAGGAGGTCTACGTCTGCGTG
ATCGAGCTCGTCAAGTGCTTCATCGAGCTGTTCCACGAGGTCGAC
AGCCCAGCCACAGTGTACCAGACCAACGGAGGAGCCGTGATTTGG
CTGCGGTACAGCATGTGGCTCCTGACTTGCCCCGTGATCCTGATC
CACCTGAGCAACCTGACCGGACTGCACGAAGAGTACAGCAAGCGG
ACCATGACCATCCTGGTGACCGACATCGGCAACATCGTGTGGGGG
ATCACAGCCGCCTTTACAAAGGGCCCCCTGAAGATCCTGTTCTTC
ATGATCGGCCTGTTCTACGGCGTGACTTGCTTCTTCCAGATCGCC
AAGGTGTATATCGAGAGCTACCACACCCTGCCCAAAGGCGTCTGC
CGGAAGATTTGCAAGATCATGGCCTACGTCTTCTTCTGCTCTTGG
CTGATGTTCCCCGTGATGTTCATCGCCGGACACGAGGGACTGGGC
CTGATCACACCTTACACCAGCGGAATCGGCCACCTGATCCTGGAT
CTGATCAGCAAGAACACTTGGGGCTTCCTGGGCCACCACCTGAGA
GTGAAGATCCACGAGCACATCCTGATCCACGGCGACATCCGGAAG
ACAACCACCATCAACGTGGCCGGCGAGAACATGGAGATCGAGACC
TTCGTCGACGAGGAGGAGGAGGGAGGAGTGGCGGCACCGGTAGTA
GCAGTGAGCAAGGGCGAGGAGCTGTTCACCGGGGTGGTGCCCATC
CTGGTCGAGCTGGACGGCGACGTAAACGGCCACAAGTTCAGCGTG
TCCGGCGAGGGCGAGGGCGATGCCACCTACGGCAAGCTGACCCTG
AAGTTCATTTGCACCACCGGCAAGCTGCCCGTGCCCTGGCCCACC
CTCGTGACCACCCTGACCTACGGCGTGCAGTGCTTCAGCCGCTAC
CCCGACCACATGAAGCAGCACGACTTCTTCAAGTCCGCCATGCCC
GAAGGCTACGTCCAGGAGCGCACCATCTTCTTCAAGGACGACGGC
AACTACAAGACCCGCGCCGAGGTGAAGTTCGAGGGCGACACCCTG
GTGAACCGCATCGAGCTGAAGGGCATCGACTTCAAGGAGGACGGC
AACATCCTGGGGCACAAGCTGGAGTACAACTACAACAGCCACAAC
GTCTATATCATGGCCGACAAGCAGAAGAACGGCATCAAGGTGAAC
TTCAAGATCCGCCACAACATCGAGG.

In some embodiments, the optogenetic fusion protein may have the amino acid sequence of SEQ ID NO: 3, below:

METAATMTHAFISAVPSAEATIRGLLSAAAVVTPAADAHGETSNA
TTAGADHGCFPHINHGTELQHKIAVGLQWFTVIVAIVQLIFYGWH
SFKATTGWEEVYVCVIELVKCFIELFHEVDSPATVYQTNGGAVIW
LRYSMWLLTCPVILIHLSNLTGLHEEYSKRTMTILVTDIGNIVWG
ITAAFTKGPLKILFFMIGLFYGVTCFFQIAKVYIESYHTLPKGVC
RKICKIMAYVFFCSWLMFPVMFIAGHEGLGLITPYTSGIGHLILD
LISKNTWGFLGHHLRVKIHEHILIHGDIRKTTTINVAGENMEIET
FVDEEEEGGVAAPVVAVSKGEELFTGVVPILVELDGDVNGHKFSV
SGEGEGDATYGKLTLKFICTTGKLPVPWPTLVTTLTYGVQCFSRY
PDHMKQHDFFKSAMPEGYVQERTIFFKDDGNYKTRAEVKFEGDTL
VNRIELKGIDFKEDGNILGHKLEYNYNSHNVYIMADKQKNGIKVN
FKIRHNIEDGSVQLADHYQQNTPIGDGPVLLPDNHYLSTQSALSK
DPNEKRDHMVLLEFVTAAGITLGMDELYK.

In some embodiments, a composition of the disclosure includes an AAV2 vector having a gene that expresses an optogenetic fusion protein (e.g., a nucleic acid encoding a protein having 98% or 99% identity to the amino acid sequence of SEQ ID NO: 3). For example, in some embodiments, a composition of the disclosure includes an AAV2 vector having a nucleic acid encoding a protein having 98% identity to the amino acid sequence of SEQ ID NO: 3. In some embodiments, a composition of the disclosure includes an AAV2 vector having a nucleic acid encoding a protein having 99% identity to the amino acid sequence of SEQ ID NO: 3.

Effective intracellular concentrations of a gene disclosed herein may be achieved via the stable expression of a vector encoding a gene (e.g., by integration into the nuclear or mitochondrial genome of a mammalian cell), such as gene that expresses an optogenetic protein, as described herein. In order to introduce such a gene into a mammalian cell, the gene can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. The genes disclosed herein can also be introduced into a mammalian cell by targeting a vector containing a polynucleotide encoding such a gene to cell membrane phospholipids. For example, vectors can be targeted to the phospholipids on the extracellular surface of the cell membrane by linking the vector molecule to a VSV-G protein, a viral protein with affinity for all cell membrane phospholipids. Stable expression of an exogenous polynucleotide in a mammalian cell can be achieved by integration of the polynucleotide containing the gene into the nuclear genome of the mammalian cell. Expression vectors for use in the compositions and methods described herein contain a polynucleotide sequence that encodes a gene as well as, e.g., additional sequence elements used for the expression of these genes and/or the integration of these polynucleotide sequences into the genome of a mammalian cell. Certain vectors that can be used include plasmids that contain regulatory sequences, such as promoter and enhancer regions, which direct gene transcription. Other useful vectors contain polynucleotide sequences that enhance the rate of translation of these genes or improve the stability or nuclear export of the mRNA that results from gene transcription. These sequence elements include, e.g., 5′ and 3′ UTR regions, an internal ribosomal entry site (IRES), and polyA in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin.

Genes described herein can be incorporated into recombinant AAV (rAAV) vectors in order to facilitate their introduction into a cell, such as a target cell, and/or for administration. rAAV vectors useful in the conjunction with the compositions and methods described herein include recombinant nucleic acid constructs that contain (1) a gene and (2) nucleic acids that facilitate and expression of the heterologous genes. The viral nucleic acids may include those sequences of AAV that are required in cis for replication and packaging (e.g., functional ITRs) of the DNA into a virion. Such rAAV vectors may also contain marker or reporter genes.

Useful rAAV vectors include those having one or more of the naturally-occurring AAV genes deleted in whole or in part, but retain functional flanking ITR sequences. The AAV ITRs may be of any serotype (e.g., derived from serotype 2 or 5) suitable for a particular application. In some embodiments, the AAV comprises two ITRs, wherein the two ITRs comprise a first ITR (ITR1) and a second ITR (ITR2), wherein ITR1 is position 5′ to the polynucleotide encoding an optogenetic fusion protein and ITR2 is position 3′ to the polynucleotide to form a cassette comprising the structure ITR1-optogenetic fusion protein-ITR2, for example the two ITRS are AAV serotype 2 ITRs.

The genes (e.g., gene encoding optogenetic protein) described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. rAAV virions useful in conjunction with the compositions and methods described herein include those derived from a variety of AAV serotypes including AAV 1, 2, 3, 4, 5, 6, 7, 8 and 9. Also useful in conjunction with the compositions and methods described herein are pseudotyped rAAV vectors. Pseudotyped vectors include AAV vectors of a given serotype pseudotyped with a capsid gene derived from a serotype other than the given serotype (e.g., AAV1, AAV3, AAV4, AAV5, AAV6, AAV7, AAV8, or AAV9, among others). For example, a representative pseudotyped vector is an AAV2 vector encoding a therapeutic protein pseudotyped with a capsid gene derived from AAV serotype 8 or AAV serotype 9. For example, in certain embodiments, AAV virions that have mutations within the virion capsid may be used to infect particular cell types more effectively than non-mutated capsid virions. For example, suitable AAV mutants may have ligand insertion mutations for the facilitation of targeting AAV to specific cell types. Other rAAV virions that can be used in methods of the invention include those capsid hybrids that are generated by molecular breeding of viruses as well as by exon shuffling.

A contemplated vector may include appropriate expression control sequences including, but not limited to, transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein processing and/or secretion. For eukaryotic cells, expression control sequences typically include a promoter, an enhancer, such as one derived from an immunoglobulin gene, SV40, cytomegalovirus, etc., and a polyadenylation sequence which may include splice donor and acceptor sites. The polyadenylation sequence generally is inserted following the transgene sequences and before the 3′ ITR sequence. In one embodiment, the bovine growth hormone polyA is used. Another regulatory component of the vector useful is an internal ribosome entry site (IRES). An IRES sequence, or other suitable systems may be used to produce more than one polypeptide from a single gene transcript. An IRES (or other suitable sequence) is used to produce a protein that contains more than one polypeptide chain or to express two different proteins from or within the same cell. An example of an IRES is the poliovirus internal ribosome entry sequence, which supports transgene expression in retinal cells.

The selection of the promoter to be employed in the vector may be made from among a wide number of constitutive or inducible promoters that can express the selected optogenetic protein in an ocular cell. In one embodiment, the promoter is cell-specific. The term “cell-specific” means that the particular promoter selected for the recombinant vector can direct expression of the selected optogenetic protein in a particular cell type. In an embodiment, the promoter is specific for expression of the optogenetic protein in retinal ganglion cells. In an embodiment, the promoter is specific for expression of the optogenetic protein in bipolar cells. For example, the light-sensitive protein may be expressed in retinal ganglion cells via a ganglion cell-specific gene promoter, for example, Thy-I.

The architecture of the ganglion cell layer (GCL) of the primate retina may also allow for targeting of specific cell types, using, for example, mechanical means. Ganglion cell bodies lie within the GCL. Near the fovea, the GCL is at its maximal thickness, and contains several layers of cell bodies. The cell bodies of different retinal ganglion cell types lie in different positions (e.g., On-type ganglion cells lie more vitreally, as observed by multielectrode recording), which may allow them to be preferentially targeted (for example, by intravitreal administration of a viral vector (e.g., an AAV that expresses an optogenetic protein). Selective targeting to On-type cells may be achieved even with a contemplated vector that includes a non-specific promoter (e.g. CAG) because the cells lie closer to the retina's surface (i.e., vitreally), and AAVs do not penetrate the retina well when delivered by intravitreal injection.

For example, significant vision restoration may be achieved by administering neurally coded stimulation (e.g., via a neural coding device) and a contemplated optogenetic vector (e.g., an AAV that expresses an optogenetic protein) that may for example, preferentially target ON-type ganglion cells. The neural coding device can deliver optogenetic stimulation (i.e., light stimulation that activates the optogenetic protein) that is specific to different ganglion cell types (e.g., stimulation that follows On-type cell neural code or stimulation that follows Off-type cell neural code). Application of On-type neurally coded stimulation to On-type ganglion cells that express an optogenetic protein may allow the On-type cells to send normal On-type visual signals to the brain, since the On-type code allows the cells to substantially mimic the normal responses of On-type cells.

Examples of constitutive promoters which may be included in the vector contemplated herein, without limitation, the CAG promoter, CMV immediate early enhancer/chicken-actin (CA) promoter-exon 1-intron 1 element, the RSV LTR promoter/enhancer, the SV40 promoter, the CMV promoter, the 381 bp CMV immediate early gene enhancer, the dihydrofolate reductase promoter, the phosphoglycerol kinase (PGK) promoter, and the 578 bp CBA promoter-exon1-intron1. For example, a contemplated promoter is a CAG promoter. In some embodiments, the CAG promoter has the nucleic acid sequence of SEQ ID NO: 6, below:

TCGAGGTGAGCCCCACGTTCTGCTTCACTCTCCCCATCTCCCCCC
CCTCCCCACCCCCAATTTTGTATTTATTTATTTTTTAATTATTTT
GTGCAGCGATGGGGGCGGGGGGGGGGGGGGGGGGCGCGCCAGGCG
GGGCGGGGCGGGGCGAGGGGGGGGGGGGGCGAGGCGGAGAGGTGC
GGCGGCAGCCAATCAGAGCGGCGCGCTCCGAAAGTTTCCTTTTAT
GGCGAGGCGGCGGCGGCGGCGGCCCTATAAAAAGCGAAGCGCGCG
GCGGGCG.

In some embodiments, vectors contemplated herein include an enhancer, such as, for example, a WPRE enhancer. In some embodiments, a WPRE enhancer has the nucleic acid sequence of SEQ ID NO: 7, below:

ATCAACCTCTGGATTACAAAATTTGTGAAAGATTGACTGGTATTC
TTAACTATGTTGCTCCTTTTACGCTATGTGGATACGCTGCTTTAA
TGCCTTTGTATCATGCTATTGCTTCCCGTATGGCTTTCATTTTCT
CCTCCTTGTATAAATCCTGGTTGCTGTCTCTTTATGAGGAGTTGT
GGCCCGTTGTCAGGCAACGTGGCGTGGTGTGCACTGTGTTTGCTG
ACGCAACCCCCACTGGTTGGGGCATTGCCACCACCTGTCAGCTCC
TTTCCGGGACTTTCGCTTTCCCCCTCCCTATTGCCACGGCGGAAC
TCATCGCCGCCTGCCTTGCCCGCTGCTGGACAGGGGCTCGGCTGT
TGGGCACTGACAATTCCGTGGTGTTGTCGGGGAAATCATCGTCCT
TTCCTTGGCTGCTCGCCTGTGTTGCCACCTGGATTCTGCGCGGGA
CGTCCTTCTGCTACGTCCCTTCGGCCCTCAATCCAGCGGACCTTC
CTTCCCGCGGCCTGCTGCCGGCTCTGCGGCCTCTTCCGCGTCTTC
GCCTTCGCCCTCAGACGAGTCGGATCTCCCTTTGGGCCGCCTCCC
CGC.

In some embodiments, vectors contemplated herein include a poly adenylation (poly(A)) element, such as, for example, an SV40 poly(A). In some embodiments, a SV40 poly(A) has the nucleic acid sequence of SEQ ID NO: 8, below:

TAAGATACATTGATGAGTTTGGACAAACCACAACTAGAATGCAGT
GAAAAAAATGCTTTATTTGTGAAATTTGTGATGCTATTGCTTTAT
TTGTAACCATTATAAGCTGCAATAAACAAGTT.

A vector contemplated herein may include an expression cassette having, for example, the nucleic acid sequence of SEQ ID NO: 9, below:

TGTAGTTAATGATTAACCCGCCATGCTACTTATCTACGTAGCCAT
GCTCTAGGAAGAGTACCATTGACGTCAATAATGACGTATGTTCCC
ATAGTAACGCCAATAGGGACTTTCCATTGACGTCAATGGGTGGAG
TATTTACGGTAAACTGCCCACTTGGCAGTACATCAAGTGTATCAT
ATGCCAAGTACGCCCCCTATTGACGTCAATGACGGTAAATGGCCC
GCCTGGCATTATGCCCAGTACATGACCTTATGGGACTTTCCTACT
TGGCAGTACATCTACGTATTAGTCATCGCTATTACCATGGTCGAG
GTGAGCCCCACGTTCTGCTTCACTCTCCCCATCTCCCCCCCCTCC
CCACCCCCAATTTTGTATTTATTTATTTTTTAATTATTTTGTGCA
GCGATGGGGGCGGGGGGGGGGGGGGGGCGCGCGCCAGGCGGGGCG
GGGCGGGGCGAGGGGCGGGGCGGGGCGAGGCGGAGAGGTGCGGCG
GCAGCCAATCAGAGCGGCGCGCTCCGAAAGTTTCCTTTTATGGCG
AGGCGGCGGCGGCGGCGGCCCTATAAAAAGCGAAGCGCGCGGCGG
GCGGGAGTCGCTGCGCGCTGCCTTCGCCCCGTGCCCCGCTCCGCC
GCCGCCTCGCGCCGCCCGCCCCGGCTCTGACTGACCGCGTTACTC
CCACAGGTGAGCGGGCGGGACGGCCCTTCTCCTCCGGGCTGTAAT
TAGCGCTTGGTTTAATGACGGCTTGTTTCTTTTCTGTGGCTGCGT
GAAAGCCTTGAGGGGCTCCGGGAGGGCCCTTTGTGCGGGGGGAGC
GGCTCGGGGCTGTCCGCGGGGGGACGGCTGCCTTCGGGGGGGACG
GGGCAGGGCGGGGTTCGGCTTCTGGCGTGTGACCGGCGGCTCTAG
AGCCTCTGCTAACCATGTTCATGCCTTCTTCTTTTTCCTACAGCT
CCTGGGCAACGTGCTGGTTATTGTGCTGTCTCATCATTTTGGCAA
AGAATTGGATCCGCCACCATGGAAACAGCCGCCACAATGACCCAC
GCCTTTATCTCAGCCGTGCCTAGCGCCGAAGCCACAATTAGAGGC
CTGCTGAGCGCCGCAGCAGTGGTGACACCAGCAGCAGACGCTCAC
GGAGAAACCTCTAACGCCACAACAGCCGGAGCCGATCACGGTTGC
TTCCCCCACATCAACCACGGAACCGAGCTGCAGCACAAGATCGCA
GTGGGACTCCAGTGGTTCACCGTGATCGTGGCTATCGTGCAGCTC
ATCTTCTACGGTTGGCACAGCTTCAAGGCCACAACCGGCTGGGAG
GAGGTCTACGTCTGCGTGATCGAGCTCGTCAAGTGCTTCATCGAG
CTGTTCCACGAGGTCGACAGCCCAGCCACAGTGTACCAGACCAAC
GGAGGAGCCGTGATTTGGCTGCGGTACAGCATGTGGCTCCTGACT
TGCCCCGTGATCCTGATCCACCTGAGCAACCTGACCGGACTGCAC
GAAGAGTACAGCAAGCGGACCATGACCATCCTGGTGACCGACATC
GGCAACATCGTGTGGGGGATCACAGCCGCCTTTACAAAGGGCCCC
CTGAAGATCCTGTTCTTCATGATCGGCCTGTTCTACGGCGTGACT
TGCTTCTTCCAGATCGCCAAGGTGTATATCGAGAGCTACCACACC
CTGCCCAAAGGCGTCTGCCGGAAGATTTGCAAGATCATGGCCTAC
GTCTTCTTCTGCTCTTGGCTGATGTTCCCCGTGATGTTCATCGCC
GGACACGAGGGACTGGGCCTGATCACACCTTACACCAGCGGAATC
GGCCACCTGATCCTGGATCTGATCAGCAAGAACACTTGGGGCTTC
CTGGGCCACCACCTGAGAGTGAAGATCCACGAGCACATCCTGATC
CACGGCGACATCCGGAAGACAACCACCATCAACGTGGCCGGCGAG
AACATGGAGATCGAGACCTTCGTCGACGAGGAGGAGGAGGGAGGA
GTGGCGGCACCGGTAGTAGCAGTGAGCAAGGGCGAGGAGCTGTTC
ACCGGGGTGGTGCCCATCCTGGTCGAGCTGGACGGCGACGTAAAC
GGCCACAAGTTCAGCGTGTCCGGCGAGGGCGAGGGCGATGCCACC
TACGGCAAGCTGACCCTGAAGTTCATTTGCACCACCGGCAAGCTG
CCCGTGCCCTGGCCCACCCTCGTGACCACCCTGACCTACGGCGTG
CAGTGCTTCAGCCGCTACCCCGACCACATGAAGCAGCACGACTTC
TTCAAGTCCGCCATGCCCGAAGGCTACGTCCAGGAGCGCACCATC
TTCTTCAAGGACGACGGCAACTACAAGACCCGCGCCGAGGTGAAG
TTCGAGGGCGACACCCTGGTGAACCGCATCGAGCTGAAGGGCATC
GACTTCAAGGAGGACGGCAACATCCTGGGGCACAAGCTGGAGTAC
AACTACAACAGCCACAACGTCTATATCATGGCCGACAAGCAGAAG
AACGGCATCAAGGTGAACTTCAAGATCCGCCACAACATCGAGGAC
GGCAGCGTGCAGCTCGCCGACCACTACCAGCAGAACACCCCCATC
GGCGACGGCCCCGTGCTGCTGCCCGACAACCACTACCTGAGCACC
CAGTCCGCCCTGAGCAAAGACCCCAACGAGAAGCGCGATCACATG
GTCCTGCTGGAGTTCGTGACCGCCGCCGGGATCACTCTCGGCATG
GACGAGCTGTACAAGTAAGAATTCGATATCAAGCTTATCGATAAT
CAACCTCTGGATTACAAAATTTGTGAAAGATTGACTGGTATTCTT
AACTATGTTGCTCCTTTTACGCTATGTGGATACGCTGCTTTAATG
CCTTTGTATCATGCTATTGCTTCCCGTATGGCTTTCATTTTCTCC
TCCTTGTATAAATCCTGGTTGCTGTCTCTTTATGAGGAGTTGTGG
CCCGTTGTCAGGCAACGTGGCGTGGTGTGCACTGTGTTTGCTGAC
GCAACCCCCACTGGTTGGGGCATTGCCACCACCTGTCAGCTCCTT
TCCGGGACTTTCGCTTTCCCCCTCCCTATTGCCACGGCGGAACTC
ATCGCCGCCTGCCTTGCCCGCTGCTGGACAGGGGCTCGGCTGTTG
GGCACTGACAATTCCGTGGTGTTGTCGGGGAAATCATCGTCCTTT
CCTTGGCTGCTCGCCTGTGTTGCCACCTGGATTCTGCGCGGGACG
TCCTTCTGCTACGTCCCTTCGGCCCTCAATCCAGCGGACCTTCCT
TCCCGCGGCCTGCTGCCGGCTCTGCGGCCTCTTCCGCGTCTTCGC
CTTCGCCCTCAGACGAGTCGGATCTCCCTTTGGGCCGCCTCCCCG
CATCGATACCGTCGACCCGGGCGGCCGCTTCGAGCAGACATGATA
AGATACATTGATGAGTTTGGACAAACCACAACTAGAATGCAGTGA
AAAAAATGCTTTATTTGTGAAATTTGTGATGCTATTGCTTTATTT
GTAACCATTATAAGCTGCAATAAACAAGTTAACAACAACAATTGC
ATTCATTTTATGTTTCAGGTTCAGGGGGAGATGTGGGAGGTTTTT
TAAAGCAAGTAAAACCTCTACAAATGTGGTAAAATCGATAAGGAT
CTTCCTAGAGCATGGCTACGTAGATAAGTAGCATGGCGGGTTAAT
CATTAACTACA.

Vectors contemplated herein include an AAV2, wherein the AAV2 includes a DNA polynucleotide encoding an optogenetic fusion protein or an RNA equivalent thereof. In some embodiments, the optogenetic protein is encoded by a nucleic acid molecule having the nucleic acid sequence of SEQ ID NO: 4 or a protein having the amino acid sequence of SEQ ID NO: 5 fused to a reporter protein. In some embodiments, the reporter protein is GFP. For example, a compound of the disclosure is exemplified by an AAV vector that has a nucleic acid sequence that is at least 90% (e.g., 91%, 92%, 93%, 94%, 95%, 96%, 97%, 98%, or 99%) identical to the nucleic acid molecule of SEQ ID NO: 1. For example, in some embodiments, the AAV vector has a nucleic acid sequence that is at least 91%, identical to the nucleic acid molecule of SEQ ID NO: 1. In some embodiments, the AAV vector has a nucleic acid sequence that is at least 92%, identical to the nucleic acid molecule of SEQ ID NO: 1. In some embodiments, the AAV vector has a nucleic acid sequence that is at least 93%, identical to the nucleic acid molecule of SEQ ID NO: 1. In some embodiments, the AAV vector has a nucleic acid sequence that is at least 94%, identical to the nucleic acid molecule of SEQ ID NO: 1. In some embodiments, the AAV vector has a nucleic acid sequence that is at least 95%, identical to the nucleic acid molecule of SEQ ID NO: 1. In some embodiments, the AAV vector has a nucleic acid sequence that is at least 96%, identical to the nucleic acid molecule of SEQ ID NO: 1. In some embodiments, the AAV vector has a nucleic acid sequence that is at least 97%, identical to the nucleic acid molecule of SEQ ID NO: 1. In some embodiments, the AAV vector has a nucleic acid sequence that is at least 98%, identical to the nucleic acid molecule of SEQ ID NO: 1. In some embodiments, the AAV vector has a nucleic acid sequence that is at least 99%, identical to the nucleic acid molecule of SEQ ID NO: 1.

In some embodiments, a AAV vector described herein comprises the nucleic acid sequence of SEQ ID NO: 1, below:

GCTGCGCGCTCGCTCGCTCACTGAGGCCGCCCGGGCAAAGCCCGG
GCGTCGGGCGACCTTTGGTCGCCCGGCCTCAGTGAGCGAGCGAGC
GCGCAGAGAGGGAGTGGCCAACTCCATCACTAGGGGTTCCTTGTA
GTTAATGATTAACCCGCCATGCTACTTATCTACGTAGCCATGCTC
TAGGAAGAGTACCATTGACGTCAATAATGACGTATGTTCCCATAG
TAACGCCAATAGGGACTTTCCATTGACGTCAATGGGTGGAGTATT
TACGGTAAACTGCCCACTTGGCAGTACATCAAGTGTATCATATGC
CAAGTACGCCCCCTATTGACGTCAATGACGGTAAATGGCCCGCCT
GGCATTATGCCCAGTACATGACCTTATGGGACTTTCCTACTTGGC
AGTACATCTACGTATTAGTCATCGCTATTACCATGGTCGAGGTGA
GCCCCACGTTCTGCTTCACTCTCCCCATCTCCCCCCCCTCCCCAC
CCCCAATTTTGTATTTATTTATTTTTTAATTATTTTGTGCAGCGA
TGGGGGCGGGGGGGGGGGGGGGGGCGCGCCAGGCGGGGCGGGGCG
GGGCGAGGGGCGGGGCGGGGCGAGGCGGAGAGGTGCGGCGGCAGC
CAATCAGAGCGGCGCGCTCCGAAAGTTTCCTTTTATGGCGAGGCG
GCGGCGGCGGCGGCCCTATAAAAAGCGAAGCGCGCGGCGGGCGGG
AGTCGCTGCGCGCTGCCTTCGCCCCGTGCCCCGCTCCGCCGCCGC
CTCGCGCCGCCCGCCCCGGCTCTGACTGACCGCGTTACTCCCACA
GGTGAGCGGGCGGGACGGCCCTTCTCCTCCGGGCTGTAATTAGCG
CTTGGTTTAATGACGGCTTGTTTCTTTTCTGTGGCTGCGTGAAAG
CCTTGAGGGGCTCCGGGAGGGCCCTTTGTGCGGGGGGAGCGGCTC
GGGGCTGTCCGCGGGGGGACGGCTGCCTTCGGGGGGGACGGGGCA
GGGCGGGGTTCGGCTTCTGGCGTGTGACCGGCGGCTCTAGAGCCT
CTGCTAACCATGTTCATGCCTTCTTCTTTTTCCTACAGCTCCTGG
GCAACGTGCTGGTTATTGTGCTGTCTCATCATTTTGGCAAAGAAT
TGGATCCGCCACCATGGAAACAGCCGCCACAATGACCCACGCCTT
TATCTCAGCCGTGCCTAGCGCCGAAGCCACAATTAGAGGCCTGCT
GAGCGCCGCAGCAGTGGTGACACCAGCAGCAGACGCTCACGGAGA
AACCTCTAACGCCACAACAGCCGGAGCCGATCACGGTTGCTTCCC
CCACATCAACCACGGAACCGAGCTGCAGCACAAGATCGCAGTGGG
ACTCCAGTGGTTCACCGTGATCGTGGCTATCGTGCAGCTCATCTT
CTACGGTTGGCACAGCTTCAAGGCCACAACCGGCTGGGAGGAGGT
CTACGTCTGCGTGATCGAGCTCGTCAAGTGCTTCATCGAGCTGTT
CCACGAGGTCGACAGCCCAGCCACAGTGTACCAGACCAACGGAGG
AGCCGTGATTTGGCTGCGGTACAGCATGTGGCTCCTGACTTGCCC
CGTGATCCTGATCCACCTGAGCAACCTGACCGGACTGCACGAAGA
GTACAGCAAGCGGACCATGACCATCCTGGTGACCGACATCGGCAA
CATCGTGTGGGGGATCACAGCCGCCTTTACAAAGGGCCCCCTGAA
GATCCTGTTCTTCATGATCGGCCTGTTCTACGGCGTGACTTGCTT
CTTCCAGATCGCCAAGGTGTATATCGAGAGCTACCACACCCTGCC
CAAAGGCGTCTGCCGGAAGATTTGCAAGATCATGGCCTACGTCTT
CTTCTGCTCTTGGCTGATGTTCCCCGTGATGTTCATCGCCGGACA
CGAGGGACTGGGCCTGATCACACCTTACACCAGCGGAATCGGCCA
CCTGATCCTGGATCTGATCAGCAAGAACACTTGGGGCTTCCTGGG
CCACCACCTGAGAGTGAAGATCCACGAGCACATCCTGATCCACGG
CGACATCCGGAAGACAACCACCATCAACGTGGCCGGCGAGAACAT
GGAGATCGAGACCTTCGTCGACGAGGAGGAGGAGGGAGGAGTGGC
GGCACCGGTAGTAGCAGTGAGCAAGGGCGAGGAGCTGTTCACCGG
GGTGGTGCCCATCCTGGTCGAGCTGGACGGCGACGTAAACGGCCA
CAAGTTCAGCGTGTCCGGCGAGGGCGAGGGCGATGCCACCTACGG
CAAGCTGACCCTGAAGTTCATTTGCACCACCGGCAAGCTGCCCGT
GCCCTGGCCCACCCTCGTGACCACCCTGACCTACGGCGTGCAGTG
CTTCAGCCGCTACCCCGACCACATGAAGCAGCACGACTTCTTCAA
GTCCGCCATGCCCGAAGGCTACGTCCAGGAGCGCACCATCTTCTT
CAAGGACGACGGCAACTACAAGACCCGCGCCGAGGTGAAGTTCGA
GGGCGACACCCTGGTGAACCGCATCGAGCTGAAGGGCATCGACTT
CAAGGAGGACGGCAACATCCTGGGGCACAAGCTGGAGTACAACTA
CAACAGCCACAACGTCTATATCATGGCCGACAAGCAGAAGAACGG
CATCAAGGTGAACTTCAAGATCCGCCACAACATCGAGGACGGCAG
CGTGCAGCTCGCCGACCACTACCAGCAGAACACCCCCATCGGCGA
CGGCCCCGTGCTGCTGCCCGACAACCACTACCTGAGCACCCAGTC
CGCCCTGAGCAAAGACCCCAACGAGAAGCGCGATCACATGGTCCT
GCTGGAGTTCGTGACCGCCGCCGGGATCACTCTCGGCATGGACGA
GCTGTACAAGTAAGAATTCGATATCAAGCTTATCGATAATCAACC
TCTGGATTACAAAATTTGTGAAAGATTGACTGGTATTCTTAACTA
TGTTGCTCCTTTTACGCTATGTGGATACGCTGCTTTAATGCCTTT
GTATCATGCTATTGCTTCCCGTATGGCTTTCATTTTCTCCTCCTT
GTATAAATCCTGGTTGCTGTCTCTTTATGAGGAGTTGTGGCCCGT
TGTCAGGCAACGTGGCGTGGTGTGCACTGTGTTTGCTGACGCAAC
CCCCACTGGTTGGGGCATTGCCACCACCTGTCAGCTCCTTTCCGG
GACTTTCGCTTTCCCCCTCCCTATTGCCACGGCGGAACTCATCGC
CGCCTGCCTTGCCCGCTGCTGGACAGGGGCTCGGCTGTTGGGCAC
TGACAATTCCGTGGTGTTGTCGGGGAAATCATCGTCCTTTCCTTG
GCTGCTCGCCTGTGTTGCCACCTGGATTCTGCGCGGGACGTCCTT
CTGCTACGTCCCTTCGGCCCTCAATCCAGCGGACCTTCCTTCCCG
CGGCCTGCTGCCGGCTCTGCGGCCTCTTCCGCGTCTTCGCCTTCG
CCCTCAGACGAGTCGGATCTCCCTTTGGGCCGCCTCCCCGCATCG
ATACCGTCGACCCGGGCGGCCGCTTCGAGCAGACATGATAAGATA
CATTGATGAGTTTGGACAAACCACAACTAGAATGCAGTGAAAAAA
ATGCTTTATTTGTGAAATTTGTGATGCTATTGCTTTATTTGTAAC
CATTATAAGCTGCAATAAACAAGTTAACAACAACAATTGCATTCA
TTTTATGTTTCAGGTTCAGGGGGAGATGTGGGAGGTTTTTTAAAG
CAAGTAAAACCTCTACAAATGTGGTAAAATCGATAAGGATCTTCC
TAGAGCATGGCTACGTAGATAAGTAGCATGGCGGGTTAATCATTA
ACTACAAGGAACCCCTAGTGATGGAGTTGGCCACTCCCTCTCTGC
GCGCTCGCTCGCTCACTGAGGCCGGGCGACCAAAGGTCGCCCGAC
GCCCGGGCTTTGCCCGGGCGGCCTCAGTGAGCGAGCGAGCGCGCA
GCTGCA.

### Pharmaceutical Compositions and Routes of Administration

Any one of the compositions described herein, such as a vector having a gene that encodes an optogenetic protein (e.g., SEQ ID NO: 5), can be formulated into pharmaceutical compositions for administration to a mammalian (e.g., a human) subject in a biologically compatible form suitable for administration in vivo. The compositions disclosed herein may be formulated in any suitable vehicle for delivery to a subject (e.g., a human). For instance, they may be formulated in a pharmaceutically acceptable suspension, dispersion, solution, or emulsion. Suitable mediums include saline and liposomal preparations. Pharmaceutically acceptable carriers may include sterile aqueous of non-aqueous solutions, suspensions, and emulsions. Recombinant human album (rAlbumin Human NF RECOMBUMIN® Prime) may also be used as a stabilizer with an AAV vector (Albumedix, Nottingham UK). Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Preservatives and other additives may also be present such as, for example, antimicrobials, antioxidants, chelating agents, and inert gases and the like. A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes. The compositions described herein may be used in the form of the free base, in the form of salts, solvates, and as prodrugs. All forms are within the methods described herein.

An optogenetic protein and cell-specific promoter for use in the target ocular cell as detailed above may be assessed for contamination by conventional methods and then formulated into a pharmaceutical composition intended for retinal injection. Such formulation involves the use of a pharmaceutically and/or physiologically acceptable vehicle or carrier, particularly one suitable for intravitreal, retinal, or subretinal injection, such as buffered saline or other buffers, e.g., HEPES, to maintain pH at appropriate physiological levels. the presence of glycerol.

According to the method of this invention for treating an ocular disorder characterized by retinal degeneration, the pharmaceutical composition described above is administered to the subject having such a blinding disease by intravitreal, retinal, or subretinal injection.

### Dosing

A vector described herein, such as, for example, an AAV2 vector that includes the nucleic acid sequence of SEQ ID NO: 9, may be administered to a human patient in an initial vector dose of about 1×10¹¹ to 1×10¹³ (e.g., about 2×10¹¹ vg/eye to about 1×10¹³ vg/eye, 3×10¹¹ vg/eye to about 1×10¹³ vg/eye, 4×10¹¹ vg/eye to about 1×10¹³ vg/eye, 5×10¹¹ vg/eye to about 1×10¹³ vg/eye, 6×10¹¹ vg/eye to about 1×10¹³ vg/eye, 7×10¹¹ vg/eye to about 1×10¹³ vg/eye, 8×10¹¹ vg/eye to about 1×10¹³ vg/eye, 9×10¹¹ vg/eye to about 1×10¹³ vg/eye, 1×10¹² vg/eye to about 1×10¹³ vg/eye, 2×10¹² vg/eye to about 1×10¹³ vg/eye, 3×10¹² vg/eye to about 1×10¹³ vg/eye, 4×10¹² vg/eye to about 1×10¹³ vg/eye, 5×10¹² vg/eye to about 1×10¹³ vg/eye, 6×10¹² vg/eye to about 1×10¹³ vg/eye, 7×10¹² vg/eye to about 1×10¹³ vg/eye, 8×10¹² vg/eye to about 1×10¹³ vg/eye, 9×10¹² vg/eye to about 1×10¹³ vg/eye). Alternatively, an AAV2 vector that includes the nucleic acid sequence of SEQ ID NO: 9 may be administered in an initial vector dose of about 1×10¹² vg/eye to about 1×10¹³ vg/eye or more. Such doses (e.g., 1×10¹² vg/eye) may produce a supralinear improvement for e.g. light sensitivity (e.g., the improvement/dose increase is not linear).

In some embodiments, the initial vector (e.g., an AAV2 vector that includes the nucleic acid sequence of SEQ ID NO: 9) dose is from about 70 μL to about 130 μL (e.g., about 80 μL to about 120 μL, about 90 μL to about 110 μL, or about 100 μL) with a concentration of about 1×10¹² to about 1×10¹⁴ vg/mL (e.g., about 2×10¹² vg/mL to about 1×10¹⁴ vg/mL, 3×10¹² vg/mL to about 1×10¹⁴ vg/mL, 4×10¹² vg/mL to about 1×10¹⁴ vg/mL, 5×10¹² vg/mL to about 1×10¹⁴ vg/mL, 6×10¹² vg/mL to about 1×10¹⁴ vg/mL, 7×10¹² vg/mL to about 1×10¹⁴ vg/mL, 8×10¹² vg/mL to about 1×10¹⁴ vg/mL, 9×10¹² vg/mL to about 1×10¹⁴ vg/mL, 1×10¹³ vg/mL to about 1×10¹⁴ vg/mL, 2×10¹³ vg/mL to about 1×10¹⁴ vg/mL, 3×10¹³ vg/mL to about 1×10¹⁴ vg/mL, 4×10¹³ vg/mL to about 1×10¹⁴ vg/mL, 5×10¹³ vg/mL to about 1×10¹⁴ vg/mL, 6×10¹³ vg/mL to about 1×10¹⁴ vg/mL, 7×10¹³ vg/mL to about 1×10¹⁴ vg/mL, 8×10¹³ vg/mL to about 1×10¹⁴ vg/mL, 9×10¹³ vg/mL).

In some embodiments, the AAV2 vector that includes the nucleic acid sequence of SEQ ID NO: 1, which may be administered to a human patient in an initial vector dose of about 1×10¹¹ to 1×10¹³ (e.g., about 2×10¹¹ vg/eye to about 1×10¹³ vg/eye, 3×10¹¹ vg/eye to about 1×10¹³ vg/eye, 4×10¹¹ vg/eye to about 1×10¹³ vg/eye, 5×10¹¹ vg/eye to about 1×10¹³ vg/eye, 6×10¹¹ vg/eye to about 1×10¹³ vg/eye, 7×10¹¹ vg/eye to about 1×10¹³ vg/eye, 8×10¹¹ vg/eye to about 1×10¹³ vg/eye, 9×10¹¹ vg/eye to about 1×10¹³ vg/eye, 1×10¹² vg/eye to about 1×10¹³ vg/eye, 2×10¹² vg/eye to about 1×10¹³ vg/eye, 3×10¹² vg/eye to about 1×10¹³ vg/eye, 4×10¹² vg/eye to about 1×10¹³ vg/eye, 5×10¹² vg/eye to about 1×10¹³ vg/eye, 6×10¹² vg/eye to about 1×10¹³ vg/eye, 7×10¹² vg/eye to about 1×10¹³ vg/eye, 8×10¹² vg/eye to about 1×10¹³ vg/eye, 9×10¹² vg/eye to about 1×10¹³ vg/eye). Alternatively, an AAV2 vector that includes the nucleic acid sequence of SEQ ID NO: 1 may be administered in an initial vector dose of about 1×10¹² vg/eye to about 1×10¹³ vg/eye or more. Such doses (e.g., 1×10¹² vg/eye) may produce a supralinear improvement for e.g. light sensitivity (e.g., the improvement/dose increase is not linear).

In some embodiments, the initial vector (e.g., an AAV2 vector that includes the nucleic acid sequence of SEQ ID NO: 1) dose is from about 70 μL to about 130 μL (e.g., about 80 μL to about 120 μL, about 90 μL to about 110 μL, or about 100 μL) with a concentration of about 1×10¹² to about 1×10¹⁴ vg/mL (e.g., about 2×10¹² vg/mL to about 1×10¹⁴ vg/mL, 3×10¹² vg/mL to about 1×10¹⁴ vg/mL, 4×10¹² vg/mL to about 1×10¹⁴ vg/mL, 5×10¹² vg/mL to about 1×10¹⁴ vg/mL, 6×10¹² vg/mL to about 1×10¹⁴ vg/mL, 7×10¹² vg/mL to about 1×10¹⁴ vg/mL, 8×10¹² vg/mL to about 1×10¹⁴ vg/mL, 9×10¹² vg/mL to about 1×10¹⁴ vg/mL, 1×10¹³ vg/mL to about 1×10¹⁴ vg/mL, 2×10¹³ vg/mL to about 1×10¹⁴ vg/mL, 3×10¹³ vg/mL to about 1×10¹⁴ vg/mL, 4×10¹³ vg/mL to about 1×10¹⁴ vg/mL, 5×10¹³ vg/mL to about 1×10¹⁴ vg/mL, 6×10¹³ vg/mL to about 1×10¹⁴ vg/mL, 7×10¹³ vg/mL to about 1×10¹⁴ vg/mL, 8×10¹³ vg/mL to about 1×10¹⁴ vg/mL, 9×10¹³ vg/mL).

In some instances, a booster dose may be desired. Such booster dosages and the need therefore can be monitored by the attending physicians, using, for example, the retinal and visual function tests and the visual behavior tests as described herein. Other similar tests may be used to determine the status of the treated subject over time. Selection of the appropriate tests may be made by the attending physician. Still alternatively, the method of this invention may also involve injection of a larger volume of virus-containing solution in a single or multiple injection to allow levels of visual function close to those found in normal retinas.

Combination therapy that includes administering to the patient light stimulation after the initial dose of an AAV2 vector that includes the nucleic acid sequence of SEQ ID NO: 9 is also contemplated For example, the light stimulation may be provided via ambient light (e.g., at levels of from about 1×10⁻⁷ mW/mm² to about 1×10⁻² mW/mm²) or with a device (e.g., at levels from about 1×10⁻⁷ mW/mm² to about 0.1 mW/mm² mW/mm²)

Alternatively, or in addition to the above, a combination therapy may include administering to the patient neurally-coded stimulation after the initial dose of Compound A (AAV2 vector that includes the nucleic acid sequence of SEQ ID NO: 9). In some embodiments, the neurally-coded light stimulation is administered at about 5×10⁻² mW/mm² to about 0.1 mW/mm², about 1×10⁻² mW/mm² to about 0.1 mW/mm², about 1×10⁻³ mW/mm² to about 0.1 mW/mm², about 1×10⁻⁴ mW/mm² to about 0.1 mW/mm², or about 1×10⁻⁵ mW/mm² to about 0.1 mW/mm².

In some embodiments, the neurally-coded stimulation includes using a light delivery device that sends light pulses in the retina's neural code. Such a stimulation may cause the ganglion cell firing to mimic that of the normal retina. For example, in some embodiments, the neurally-coded stimulation includes using a light delivery device that sends light pulses in the retina's neural code, causing the ganglion cell firing to mimic that of the normal retina.

The stimulation could use any suitable mechanism, and can include optogenetic stimulators or other light-delivery stimulators, as described in U.S. Pat. No. 9,220,634, which is incorporated herein in its entirety by reference.

The output interface of the stimulation may be a digital light processing (DLP) device. This DLP device would output pulses of light. The pulses of light would then drive the optogenetic protein in the ganglion cells, causing the ganglion cells to fire as an encoder specifies. In this example, the output interface functions as follows: the output of an encoder is sent from a processing unit to the output interface (e.g., DLP). The output interface then converts the binary data, which represents action potential times, into light pulses, using a digital micromirror device (DMD) that is paired with a light emitting diode (LED). The DMD may be a grid of mirrors whose position can be switched with high temporal and spatial resolution. When an encoder dictate that the ganglion cell at position (x,y) should fire an action potential, the mirror at position (x,y) on the device is switched to the ‘on’ position for a brief period (e.g., millisecond-timescale), and then switched back to the ‘off’ position. This reflects light from the LED onto the retina for a brief period, causing a light pulse at position (x,y). This light pulse drives the retinal ganglion cell at position (x,y) to fire.

In one embodiment, the stimulation output interface is a digital light processing (DLP) device as described above. The standard light source on the DLP device may be replaced with a high intensity LED, intense enough to activate an optogenetic protein, such as SEQ ID NO: 5. As mentioned above, the DLP may contain a digital micromirror device (DMD) (DLPl 3010LC, Texas Instruments, Dallas, TX), which consists of a grid of mirrors, each of which can be switched to reflect the light from the LED onto the retina when the retinal ganglion cell at that location should fire. Data is sent from an encoding device that uses the retina's neural code to the output interface over a High Definition Multimedia Interface (HDMI, 22 MB/sec). The position of each mirror on the DMD is controlled with high temporal resolution (e.g., when an encoder dictates that a ganglion cell should fire an action potential, the mirror at the corresponding location is switched to the ‘on’ position for a brief time period (e.g., 1.4 ms). The mirror switching states causes the device to output a pulse of light to the corresponding location, which drives the targeted retinal ganglion cell to fire an action potential. The mirror switching time may be shorter or longer, for example from 0.1 ms to 10 ms, depending on the amount of light required to activate the cell. In this embodiment, the array of mirrors on the DMD may be 480 by 320 mirrors, and thus be capable of targeting over 150,000 locations (e.g., cells) independently. The DLP could also have more mirrors, e.g., 1024 by 768 mirrors, as in the case of the DLP5500A (Texas Instruments, Dallas, TX), and thus could stimulate many more locations independently. Data transfer between an encoding device and the interface follows standard specifications, as laid out in Texas Instruments Application Report DLPA021—January 2010—“Using the DLP Pico 2.0 Kit for Structured Light Applications.”

The DLP is one example of a potential output interface. The output interface could also be implemented using any device capable of activating the optogenetic protein. Examples include, but is not limited to, Digital micromirror devices; LED arrays; Spatial light modulators; Fiber optics; Lasers; Xenon lamps; Scanning mirrors; Liquid-crystal displays (LCDs), and the combinations thereof. (Golan L, et al 2009; Grossman N et al., 2010)

Alternatively, or in addition to the above, a combination therapy may include administering a corticosteroid (e.g., prednisone, prednisolone, cortisone, methylprednisolone, dexamethasone, betamethasone, or hydrocortisone), for example administered orally. In some embodiments, the corticosteroid is prednisone/prednisolone.

The corticosteroid may be administered to the patient 1 to 3 (e.g., 2) days prior to the intravitreal administration of the AAV2 vector that includes the nucleic acid sequence of SEQ ID NO: 1. For example, in some embodiments, the patient is administered an oral corticosteroid 1 to 3 days prior to the intravitreal administration of SEQ ID NO: 1.

In some embodiments, the oral prednisone/prednisolone is administered at 1 mg/kg. In some embodiments, the oral prednisone/prednisolone is administered at this dose for 7 days after the injection including the injection day (e.g., the day SEQ ID NO: 1 is administered) for a total of 10 days. For example, in some embodiments, the corticosteroid is oral prednisone/prednisolone administered 1 mg/kg prednisone/prednisolone for 1-3 (e.g., 2) days prior to injection or on day of injection and optionally continuing at this dose for 7 days after the injection including the injection day a total of 10 days.

Alternatively, or in addition to the above, a combination therapy may include administering valacyclovir or acyclovir. Valacyclovir may be administered at 1000 mg/day or acyclovir 400 mg twice a day orally starting 3-7 days prior to the intravitreal administration. For example, in some embodiments, valacyclovir may be administered at 1000 mg/day orally starting 3-7 days prior to the intravitreal administration. In some embodiments, acyclovir may be administered at 400 mg twice a day orally starting 3-7 days prior to the intravitreal administration.

### Methods of Treatment

Provided herein is a method of treating a retinal degenerative disorder in a human patient in need thereof comprising administering intravitreally to the patients eye a vector dose of an effective amount of a AAV2 vector having a gene that expresses an optogenetic protein, e.g., where the vector comprises a sequence having e.g., 95% to 100% identity over the length of SEQ ID NO: 1 Contemplated retinal degenerative diseases include retinitis pigmentosa (RP), age-related macular degeneration, Usher syndrome, Stargardt macular dystrophy, Leber congenital amaurosis and Bardet-Biedl syndrome. Also contemplated are retinal disorders including retinal detachment and retinal vessel occlusion.

Retinitis pigmentosa includes autosomal recessive inherited retinitis pigmentosa as well as autosomal dominant inherited retinitis pigmentosa and X-chromosome recessive inherited retinitis pigmentosa. The most common retinitis pigmentosa is the type showing autosomal recessive inheritance, which accounts for about 35% of the total. The next most common is the type showing autosomal dominant inheritance, which accounts for 10% of the total. The least common is the type showing X-linked inheritance (X-chromosome recessive inheritance), which accounts for about 5% of the total.

Diseases in which retinal degeneration occurs as a complication are also contemplated in the disclosure herein. Such diseases include: Snowflake vitreoretinal degeneration; Choroidal neovasculatization caused by adult-onset foveomacular dystrophy; Bietti crystalline corneoretinal dystrophy; and diabetic retinopathy. A partial list of diseases in which retinal degeneration occurs as a symptom include: Aceruloplasminemia; Adrenoleukodystrophy; Alstrom disease; Alstrom Syndrome; Asphyxiating Thoracic Dystrophy; Bonneman-Meinecke-Reich syndrome; Bonnemann-Meinecke-Reich syndrome; CDG syndrome type IA; Chorioretinopathy dominant form-microcephaly; Choroideremia-hypopituitarism; Congenital disorder of glycosylation type IA; Congenital Disorders of Glycosylation Type Ia; Cystinosis; Hypotrichosis, syndactyly and retinal degeneration; Jeune syndrome; Mucolipidosis IV; Mucolipidosis type 4; Mucopolysaccharidoses; Muscle-eye-brain syndrome; Neonatal ALD; Olivopontocerebellar atrophy type 3; Osteopetrosis, autosomal recessive 4; Pigmentary retinopathy; Pseudoadrenoleukodystrophy; Retinoschisis, X-linked; Retinoschisisl, X-linked, Juvenile; Santavuori Disease; Spastic paraplegia, autosomal recessive; and Werner syndrome. The present methods can be used to treat any mammalian subject who has RP. Any of the compositions described herein may be used in a method of treatment, for example, in a method of improving light sensitivity in a subject in need thereof. Such treatment may, for example, obtain a desired therapeutic effect in terms of partially or completely curing a disease and/or adverse effect attributed to the disease.

The human patients may have advanced stage blindness due to the RP, or mild, moderate, or severe visual impairment. For example, a contemplated patient may have bare light perception (BLP) or no light perception (NLP) at baseline (i.e., before treatment).

Contemplated patients may have early stage disease and/or visual acuity no better than 20/200 or about 20/4200 at baseline, for example, a patient may have a visual acuity of about 20/200 at baseline. Contemplated patients may have visual acuity no better than the ability to count fingers at baseline, For example, in some embodiments, the patient has visual acuity no better than 20/200 at baseline or less than visual acuity to count fingers at baseline. Contemplated patients may have visual acuity no better than 20/60 (or e.g., about 20/60 to about 20/200 or more, and/or for example, having a baseline visual field test indicating mild, moderate and/or severe (e.g., tunnel vision) visual field impairment.

The beneficial treatment effects of the compositions and methods described herein, such as the ability of Compound A described herein, to cause the ganglion cell firing to mimic that of the normal retina may manifest clinically in a variety of ways. For example, in some embodiments, following an initial dose of Compound A the patient has improved light sensitivity whose manifestation includes an increase in amplitude of electroretinogram (ERG) response and/or Visually Evoked Potential (VEP) as compared to baseline. In some embodiments, the patient has improved light sensitivity that manifests as an increase in the amplitude of ERG or VEP response as compared to baseline at 4 months or more (e.g., 5 months, 6 months, 7 months, 8 months, 9 months, 10 months, 11 months, or 1 year, or longer) after the initial dose of Compound A.

In some embodiments, following an initial dose of Compound A, the patient's light perception threshold has decreased, as measured by a staircase or binary search procedure. In some embodiments, the patient's light perception threshold has decreased, as measured by a staircase or binary search procedure as compared to baseline at 4 months or more (e.g., 5 months, 6 months, 7 months, 8 months, 9 months, 10 months, 11 months, or 1 year, or longer) after the initial dose of Compound A.

In some embodiments, following an initial dose of Compound A, the patient's ability to detect motion and/or direction of motion has increased as measured by a standard two-alternative forced choice paradigm. In some embodiments, the patient's ability to detect motion and/or direction of motion has increased as measured by a standard two-alternative forced choice paradigm as compared to baseline at 3 months or more (e.g., 4 months, 5 months, 6 months, 7 months, 8 months, 9 months, 10 months, 11 months, or 1 year, or longer) after the initial dose of Compound A.

In some embodiments, following an initial dose of Compound A: 1, the patient's shape detection ability has increased as measured by a standard two-alternative forced choice paradigm. In some embodiments, the patient's shape detection ability has increased as measured by a standard two-alternative forced choice paradigm as compared to baseline at 4 months or more (e.g., 5 months, 6 months, 7 months, 8 months, 9 months, 10 months, 11 months, or 1 year, or longer) after the initial dose of Compound A.

In some embodiments, following an initial dose of Compound A, the patient has an increased ability to detect and/or distinguish colors. For example, in some embodiments, following an initial dose of Compound A, the patient has an increased ability to detect colors. In some embodiments, following an initial dose of Compound A, the patient has an increased ability to distinguish colors. In some embodiments, the patient has an increased ability to detect colors compared to baseline at 4 months or more (e.g., 5 months, 6 months, 7 months, 8 months, 9 months, 10 months, 11 months, or 1 year, or longer) after the initial dose of Compound A. In some embodiments, the patient has an increased ability to distinguish colors compared to baseline at 4 months or more (e.g., 5 months, 6 months, 7 months, 8 months, 9 months, 10 months, 11 months, or 1 year, or longer) after the initial dose of Compound A.

Other methods to measure clinical efficacy include evaluation of the flash visual evoked response (VEP), the pupillary light reflex (PLR), ERG (including bilateral full-field ERG), and nystagmus testing. International Society for Clinical Electrophysiology of Vision standard guideline may be followed for the analyses. Pupil responses may be recorded simultaneously in both eyes. Nystagmus may be characterized qualitatively and quantitatively by analysis of motion paths in videos taken at baseline and at various desired time points post-treatment. Interpupillary distances may be measured directly from video frames. Subjective measures include, but not limited to, standard tests of visual acuity (VA), kinetic visual field, and mobility testing to assess the ability of the subjects to navigate an obstacle course. For mobility testing, different mazes may be used each time the test is performed and number of obstacles avoided or hit, number of landmarks identified and time spent in the maze can then be assessed.

The compositions and methods described herein may provide beneficial clinical effects that may last for extended periods of time. For example, after administering an initial dose of an AAV2 vector having a gene that expresses an optogenetic protein, wherein the vector includes the nucleic acid sequence of SEQ ID NO: 1, a patient having RP may exhibit a therapeutic benefit, as compared to baseline at 3 months or more (e.g., 4 months, 5 months, 6 months, 7 months, 8 months, 9 months, 10 months, 11 months, or 1 year, or longer) post-administration. For example, in some embodiments, after administering an initial dose of such a vector having a gene that expresses an optogenetic protein, a patient having RP may exhibit a therapeutic benefit, as compared to baseline at 4, 5, 6 or 7 months or more post-administration.

### Definitions

The features and other details of the disclosure will now be more particularly described. Certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

As used herein, the term “adeno-associated virus” (AAV) refers to a vector derived from an adeno-associated virus serotype, including without limitation, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16. AAV vectors can have one or more of the AAV wild-type genes deleted in whole or part, e.g., the rep and/or cap genes, but retain functional flanking inverted terminal repeat (ITR) sequences. Functional ITR sequences promote the rescue, replication, and packaging of the AAV virion. Thus, an AAV vector is defined herein to include at least those sequences required in cis for replication and packaging (e.g., functional ITRs) of the virus. ITRs do not need to be the wild-type polynucleotide sequences and may be altered, e.g., by the insertion, deletion, or substitution of nucleotides, so long as the sequences provide for functional rescue, replication, and packaging. AAV expression vectors are constructed using known techniques to at least provide as operatively linked components in the direction of transcription, control elements including a transcriptional initiation region, the DNA of interest (e.g., a vector having a gene that encodes an optogenetic protein (e.g., SEQ ID NO: 5) of the disclosure) and a transcriptional termination region. The terms “adeno-associated virus inverted terminal repeats” and “AAV ITRs” refer to art-recognized regions flanking each end of the AAV genome which function together in cis as origins of DNA replication and as packaging signals for the virus. AAV ITRs, together with the AAV rep coding region, provide for the efficient excision and integration of a polynucleotide sequence interposed between two flanking ITRs into a mammalian genome. The polynucleotide sequences of AAV ITR regions are known. As used herein, an “AAV ITR” does not necessarily include the wild-type polynucleotide sequence, which may be altered, e.g., by the insertion, deletion or substitution of nucleotides. Additionally, the AAV ITR may be derived from any of several AAV serotypes, including without limitation AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16, among others. Furthermore, 5′ and 3′ ITRs which flank a selected polynucleotide sequence in an AAV vector need not be identical or derived from the same AAV serotype or isolate, so long as they function as intended, e.g., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.EB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16, among others.

As used herein, a “combination therapy,” “administered in combination,” or “further administering” means that two (or more) different agents or treatments are administered to a subject as part of a defined treatment regimen for a particular disease or condition. The treatment regimen defines the doses and periodicity of administration of each agent such that the effects of the separate agents or treatments on the subject overlap. In some embodiments, the delivery of the two or more agents or treatments is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic treatment of the combination therapy may be light stimulation. In some embodiments, the combination therapy includes light stimulation.

Throughout the specification and claims, the word “comprise,” or variations such as “comprises” or “comprising,” will be understood to imply the inclusion of a stated word or group of words but not the exclusion of any other word or group of words.

As used herein, “Compound A” refers to the nucleic acid molecule having the nucleic acid sequence of SEQ ID NO: 1.

As used herein, the term “dose” refers to the quantity of a therapeutic agent, such as a viral vector described herein, that is administered to a subject at a particular instant for the treatment of a disorder, such as to treat or ameliorate one or more symptoms of retinitis pigmentosa. A therapeutic agent as described herein may be administered in a single dose or in multiple doses over the course of a treatment period. In each case, the therapeutic agent may be administered using one or more unit dosage forms of the therapeutic agent, a term that refers to a one or more discrete compositions containing a therapeutic agent that collectively constitute a single dose of the agent.

As used herein, the terms “effective amount,” “therapeutically effective amount,” and the like, when used in reference to a composition described herein, such as a vector having a gene that encodes an optogenetic protein (e.g., SEQ ID NO: 5), refer to a quantity sufficient to, when administered to a subject, including a mammal (e.g., a human), effect beneficial or desired results (e.g., expression of an optogenetic protein), which may include clinical results. For example, an effective amount of one or more composition described herein (e.g., a vector having a gene that encodes an optogenetic protein (e.g., SEQ ID NO: 5)) may achieve expression of a protein of interest as compared to the expression of said protein without administration of the composition of interest. An “effective amount,” “therapeutically effective amount,” and the like, of a composition, such as a vector having a gene that encodes an optogenetic protein (e.g., SEQ ID NO: 5), also include an amount that results in a beneficial or desired result in a subject as compared to a control.

The term “pharmaceutically acceptable” means safe for administration to a mammal, such as a human. In some embodiments, a pharmaceutically acceptable composition is approved by a regulatory agency of the Federal government or a state government or is listed in the U.S. Pharmacopeia or any other generally recognized pharmacopeia for use in animals (e.g., humans). As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region (e.g., a transgene)” is a transcriptional control region that is not normally associated with the coding region in nature.

The terms “pharmaceutically acceptable carrier” or “pharmaceutically acceptable excipient,” as used herein, refer interchangeably to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions together with one or more pharmaceutically acceptable excipients.

It is to be understood that the present disclosure also provides pharmaceutical compositions including any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The quantity of active ingredient in a unit dose of composition is an effective amount and is varied according to the particular treatment involved. One skilled in the art will appreciate that it is sometimes necessary to make routine variations to the dosage depending on the age and condition of the subject. The dosage will also depend on the route of administration. A variety of routes are contemplated, including intravitreally, oral, pulmonary, rectal, parenteral, transdermal, subcutaneous, intravenous, intramuscular, intraperitoneal, inhalational, buccal, sublingual, intrapleural, intrathecal, intranasal, and the like. In some embodiments, the composition is administered intravitreally. In one embodiment, the active compound is mixed under sterile conditions with a pharmaceutically acceptable carrier, and with any preservatives, buffers, or propellants that are required.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers including excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Further, compositions may include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

As used herein, the term “promoter” refers to a recognition site on DNA that is bound by an RNA polymerase. The polymerase drives transcription of a transgene. Exemplary promoters suitable for use with the compositions and methods described herein are described herein, such as a CAG promoter. Additionally, the term “promoter” may refer to a synthetic promoter, such as a regulatory DNA sequence that doe does not occur naturally in a biological system. Synthetic promoters contain parts of naturally occurring promoters combined with polynucleotide sequences that do not occur in nature and can be optimized to express recombinant DNA.

As used herein, the term “optogenetic protein” refers to a proteins, such as opsins, that are light-gated ion channels or pumps that absorb light at specific wavelengths. Upon activation by light, these channels and pumps respond by opening or closing, which conducts the flow of ions into or out of a cell. Different optogenetic proteins respond to different wavelengths of light, such as, for example, blue or yellow light. Furthermore, in addition to the optogenetic proteins found in nature, the disclosure herein includes genetically engineered optogenetic proteins, such as those which have a point mutation to alter the absorption spectrum and/or to add a signal in order to direct trafficking within a cell, such that an engineered optogenetic protein may localize to a specific location within a cell (e.g., cell membrane). An optogenetic protein may include, but is not limited to SEQ ID NO: 5. Any suitable optogenetic protein may be used in the methods described herein, including any now known or later discovered/engineered optogenetic protein.

As used herein, a “retinal degenerative disease” refers to any disease caused by degeneration of the retina, and examples thereof include, for example, retinitis pigmentosa (RP), age-related macular degeneration, Usher syndrome, Stargardt macular dystrophy, Leber congenital amaurosis and Bardet-Biedl syndrome, retinal detachment, and retinal vessel occlusion.

As used herein, “retinitis pigmentosa” is a hereditary disease with abnormalities in the retina, in which the photoreceptor and pigment epithelial cells of the retina are extensively degenerated. In the retinitis pigmentosa, three symptoms generally appear: night blindness (difficulty seeing things in the dark), narrowing of the visual field (narrow vision), and decreased visual acuity. The degeneration of only rod cells among the photoreceptor cells is called rod dystrophy, while the degeneration of both rod cells and cone cells, among the photoreceptor cells, is called rod cone dystrophy. As used herein, the “retinitis pigmentosa” includes autosomal recessive inherited retinitis pigmentosa as well as autosomal dominant inherited retinitis pigmentosa and X-chromosome recessive inherited retinitis pigmentosa.

A “subject” may include any animal, including mammals, mice, rats, other rodents, rabbits, dogs, cats, swine, cattle, sheep, horses, or non-human primates, and most preferably humans. The compositions of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, non-human primates, and the like).

As used herein, the terms “transduction” and “transduce” refer to a method of introducing a viral vector construct or a part thereof into a cell and subsequent expression of a transgene encoded by the vector construct or part thereof in the cell.

As used herein, “transfection” refers to any of a wide variety of techniques commonly used for the introduction of exogenous DNA into a prokaryotic or eukaryotic host cell e.g., electroporation, lipofection, calcium-phosphate precipitation, diethylaminoethyl (DEAE)-dextran transfection, NUCLEOFECTION™, squeeze-poration, sonoporation, optical transfection, MAGNETOFECTION™, impalefection, and the like.

The terms “treat,” “treatment,” “treating,” and the like are used herein to generally mean obtaining a desired pharmacological and/or physiological effect. The effect may be therapeutic in terms of partially or completely curing a disease and/or adverse effect attributed to the disease. The term “treatment” as used herein covers any treatment of a disease in a mammal, particularly a human, and includes: (a) inhibiting the disease, e.g., preventing the disease from increasing in severity or scope; (b) relieving the disease, e.g., causing partial or complete amelioration of the disease; or (c) preventing relapse of the disease, e.g., preventing the disease from returning to an active state following previous successful treatment of symptoms of the disease or treatment of the disease.

As used herein, the term “vector” includes a nucleic acid vector, e.g., a DNA vector, such as a plasmid, an RNA vector, or another suitable replicon (e.g., viral vector). A variety of vectors have been developed for the delivery of polynucleotides encoding exogenous polynucleotides or proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of heterologous nucleic acid materials (e.g., a vector having a gene that encodes an optogenetic protein (e.g., SEQ ID NO: 5)) in a mammalian cell. Certain vectors that can be used for the expression of the genes described herein include plasmids that contain regulatory sequences, such as promoter and enhancer regions, which direct gene transcription. Other useful vectors for expression of gene agents disclosed herein contain polynucleotide sequences that enhance the rate of translation of these polynucleotides or improve the stability or nuclear export of the RNA that results from gene transcription. These sequence elements include, e.g., 5′ and 3′ untranslated regions, an IRES, and polyA in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

### EXAMPLES

The disclosure is further illustrated by the following examples. The examples are provided for illustrative purposes only, and are not to be construed as limiting the scope or content of the disclosure in any way.

### Example 1: Safety and Efficacy of Compound A (SEQ ID NO: 1) for an Optogenetic Gene Therapy

Preclinical efficacy and safety data is obtained using a vector having a gene that expresses an optogenetic fusion protein (e.g., an AAV2 vector including a nucleic acid molecule having the nucleic acid sequence of SEQ ID NO: 2 or a nucleic acid encoding a protein having the amino acid sequence of SEQ ID NO: 3 as the optogenetic fusion protein). Efficacy was assessed in mice in a dose dependent manner using electroretinograms (ERGs). Safety was assessed in rats, nonhuman primates, and mice, using several tests, including immunohistochemical analyses and cell counts (rats), electroretinograms (nonhuman primates), and ocular toxicology assays (mice). The results showed that SEQ ID NO: 3-expressing vectors were efficacious over a broad range of vector doses and stimulating light intensities, and were well tolerated: no test article-related findings were observed in the anatomical and electrophysiological assays performed.

### Materials and Methods

### Vector Injections

All vectors were prepared in a balanced salt solution (BSS) with 0.014% Tween 20 and delivered to the eye by intravitreal injection. For rodents, animals were anesthetized with intraperitoneal ketamine/xylazine (72 mg/kg ketamine and 4 mg/kg xylazine for mouse, and 80 mg/kg ketamine and 10 mg/kg xylazine for rat), and the pupil was dilated with an atropine sulfate ophthalmic solution (1%). Using a Hamilton syringe under a dissecting microscope, the needle was passed through the sclera into the vitreous cavity. The injected volume was 1 μL for mouse and 4 μL for rat. For nonhuman primates, animals were anesthetized with a mixture of ketamine/dexmedetomidine (5-10 mg/kg ketamine and 0.01-0.02 mg/kg dexmedetomidine) and then maintained with inhaled isoflurane/oxygen mixture. Pupils were dilated with 1% atropine sulfate, 2.5% phenylephrine hydrochloride, applied topically. The vector was injected intravitreally using a 3/10 cc U-100 insulin syringe with a 30 Gauge needle. The injected volume was 80 μL to 100 μL. All animal experiments and procedures were performed according to the guidelines approved by the Institutional Animal Care and Use Committees (IACUC).

### Electroretinography (ERG)

For mice, animals were anesthetized with intraperitoneal ketamine/xylazine (72 mg/kg ketamine and 4 mg/kg xylazine), and the pupil was dilated with 1% atropine sulfate, 2.5% phenylephrine hydrochloride). To perform the ERG, a tungsten-wire electrode was placed on the corneal surface of the recorded eye and referenced to an electrode in the mouth. Visual stimuli were delivered with an LED stimulator with a 505 nm peak wavelength. The stimulator was placed 1.7 cm away from the cornea, subtending a visual angle of approximately 100 degrees, with a peak intensity of 0.06 mW/mm². The stimulation was delivered as pulsed light, periodic at 10 Hz, pulse width at 11.2 ms. Data collection was carried out with the Espion E³ electroretinography console (Diagnosys LLC, Lowell, MA).

For nonhuman primates, animals were anesthetized with a mixture of ketamine (5 mg/kg-10 mg/kg)/dexmedetomidine (0.01 mg/kg-0.02 mg/kg). Pupils were dilated with topical agents (1% atropine sulfate, 2.5% phenylephrine hydrochloride). To perform the ERG, a tungsten-wire electrode was placed on the corneal surface of the eye being tested, and referenced to a needle electrode placed in the scalp. Visual stimuli were delivered with a mini-ganzfeld stimulator placed close to the recorded eye. For the Naka-Rushton fit, photopic ERG stimulation was used following (Joshi, Ly, and Viswanathan, 2017): 5 ms red light ganzfeld flashes ranging from 0.00625 to 1.6 cd·s/m² on a constant 7 cd/m² blue background.

### Stimulation for Light Safety

Light stimulation was performed in twelve 2 hour to 2.5 hour sessions over a period of 6 weeks to 8 weeks. The light was at an intensity of 0.1 mW/mm² (a peak wavelength of 505 nm). The light was delivered in pulses with a pulse width of 5 ms, as it would be if neurally-coded stimuli were used (Nirenberg and Pandarinath 2012; Yan et al., 2016). The stimulus subtending a visual angle of approximately 60 degrees covered a large area of central retina, 4 mm diameter (Yan et al., 2016). In each session, animals were anesthetized with Isoflurane (99.9%) to a depth that minimized eye movements. Each animal was placed on its left side, with its right eye illuminated by the stimulus. The pupil was dilated with an atropine sulfate ophthalmic solution (1%) and the eye was kept wet with artificial tears applied regularly (every 7 min). The left eye was left untreated. Between sessions, the animals were exposed to normal room light with standard day/night cycles, as is standard in a rodent animal housing facility. Two to 4 weeks after the sessions were completed the animals were euthanized and the retinas removed for examination.

### Histological Analysis

For wholemount retinas, eyes were removed and fixed in 4% paraformaldehyde in PBS for 30 minutes. Retinas were dissected and fixed overnight in 4% paraformaldehyde. Autofluorescence was quenched with 1% sodium borohydride in PBS for 5 minutes. The retinas were permeabilized and blocked with 5% normal donkey serum (NDS), 1% bovine serum albumin (BSA) in PBS with 0.3% TritonX-100 for 1 hour. The retinas were then labeled with rabbit-anti GFP Alexa Fluor 555 1:200 (Invitrogen-Molecular Probes, Life Technologies, Carlsbad, CA) overnight in 5% NDS, 1% BSA in PBS or with fluorescein peanut agglutinin (FITC PNA) 1:500 in 2% BSA (Vector Laboratories, Burlington, CA) in PBS for 15 minutes. Then, the retinas were washed five times in PBS and incubated for 1.5 hours at room temperature with the Alexa Fluor-647 donkey anti-goat IgG 1:100 (Invitrogen-Molecular Probes, Life Technologies, Carlsbad, CA). The retinas were thoroughly washed in PBS and mounted.

When processed as sections, eyes were fixed in 4% paraformaldehyde. After one hour, cornea and lens were removed without disturbing the retina. The retinas were further fixed for additional 2-3 hours at room temperature. The eyecups were rinsed with PBS and cryoprotected by 30% sucrose/PBS for 4 hours at room temperature, then embedded in cryostat compound (Tissue TEK OCT, Sakura Finetek USA, Inc., Torrance, CA) and frozen at −80° C. Retinas were sectioned perpendicularly from dorsal to ventral at 12 μm thickness. For immunohistochemistry, retinal sections were rinsed in PBS and incubated in 0.3% Triton X-100 in PBS for 15 min, then blocked in 5% BSA in PBS for 1 hour at room temperature. Sections were then incubated with anti-Brn3a (1:500, Santa Cruz, sc-31984) and anti-GFP (1:200 dilution, Life Technologies, A11122) at room temperature overnight. They were washed with PBS three times, followed by incubating with IgG secondary antibodies tagged with Alexa-594 and Alexa-488 (1:500 dilution, Molecular Probes, Eugene OR) at room temperature for two hours, then washed with PBS. Sections were mounted with Vectashield Mounting Medium for Fluorescence (Vector lab, H-10400, Burlingame, CA) and cover slipped.

### Multi-Electrode Array Recording

Electrophysiological recordings were obtained in vitro from isolated retinas. Briefly, the anterior portion of the eye and vitreous were removed immediately after enucleation, and the eyecup was placed in Ringer's solution and stored in darkness for at least 20 min before dissection. Under dim red light illumination, pieces of retina 1.5-3 mm in diameter were cut from central regions and placed onto a multi-electrode array for recording. The Ringer's solution was bubbled with 95% 02 and 5% CO₂ and maintained at 35° C. to 36° C., pH 7.4. The stimulation and recording of retinal ganglion cells was performed, as in Nirenberg and Pandarinath (2012). Spike waveforms were recorded using a Plexon Instruments Multichannel Neuronal Acquisition Processor (Dallas, TX). A standard spike sorting method was used to identify individual cells as in ref. (Nirenberg and Pandarinath 2012).

### Ocular Toxicology Assays

Before injections, an ocular examination was performed. If ocular findings were present, the animal was excluded from the study. Ocular examinations were also performed on Week 2, 4, 8, and 12 post-injection by a licensed veterinary ophthalmologist. A slit lamp was used to assess anterior segments including cornea, iris, and lens. An indirect ophthalmoscope was used to assess the posterior segments including vitreous chambers and retinas. All observations were made in a masked fashion. There were two sacrifice time points: Week 4 and Week 12. At scheduled necropsies, eyes of animals were collected in 4% paraformaldehyde. Following sufficient time in fixation, the tissues were trimmed, embedded in paraffin, sectioned, and stained with hematoxylin and eosin, and slides were examined. Immunohistochemical stains for green fluorescent protein were also examined to verify the transgene expression.

### Results

### Compound a Produced Reliable ERG Responses in Blind Mice

To test the efficacy, photopic ERGs were performed in adult rd1 mice, a widely used model for retinal degenerative disease (Farber et al., 1994; Grimm et al., 2004; Hackam et al., 2004; Lagali et al., 2008; Thyragaraian et al., 2010), which has an earlier onset and severe retinal degeneration than rd10 (Pennesi et al., 2012). Briefly, the photopic ERG response in normal animals is comprised of three components: the a-wave, which corresponds to photoreceptor signaling, the b-wave, which corresponds to bipolar cell signaling, and the photopic negative response (PhNR), which corresponds to ganglion cell signaling (Viswanathan et al., 1999). In rd1 animals, which lose photoreceptor outer segments by 8 weeks of age (Grimm et al., 2004; Nirenberg and Pandarinath 2012), none of these components are present (Nishiguchi et al, 2015). If these animals are engineered to express an optogenetic protein in their ganglion cells, the prediction is that a PhNR-like wave would emerge, reflecting the newly-created optogenetic activity of the ganglion cells. ERGs were conducted on Compound A-treated eyes to test for the presence of a newly-created PhNR-like wave.

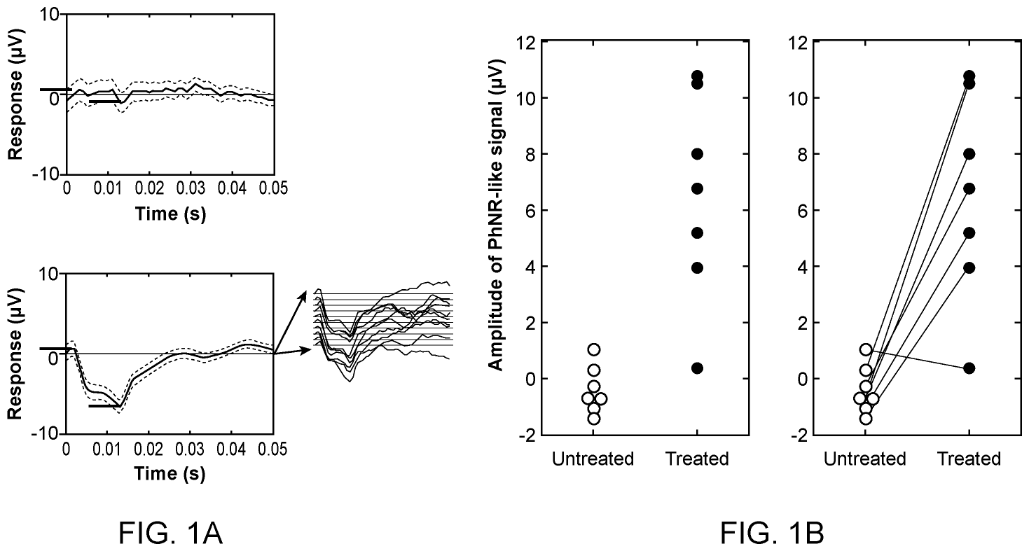

Seven rd1 mice received Compound A by intravitreal injection into one eye at a dose of 5×10⁸ vg/eye, comparable to a dose of 5×10¹¹ vg/eye in humans Outcomes were measured at 10 weeks post injection, when the Compound A expression was expected to have peaked. The untreated eye of each animal served as the control. The results showed that six of the seven animals produced reliable light-evoked ERG responses (PhNR-like waves) in their treated eyes; this is in contrast to the ERGs produced by the untreated eyes of the same rd1 animals, which showed flat ERG responses (FIGS. 1A-1B). The results were significant at both a group level (p<0.001, unpaired t-test comparing the magnitude of the ERG signal from treated eyes with those from the untreated eyes, and at an individual level (p<0.004, paired t-test, comparing, for each animal, its treated eye with its untreated counterpart).

The Magnitude of the ERG Response was Dose-Dependent, Both with Respect to Viral Dose and Light Dose

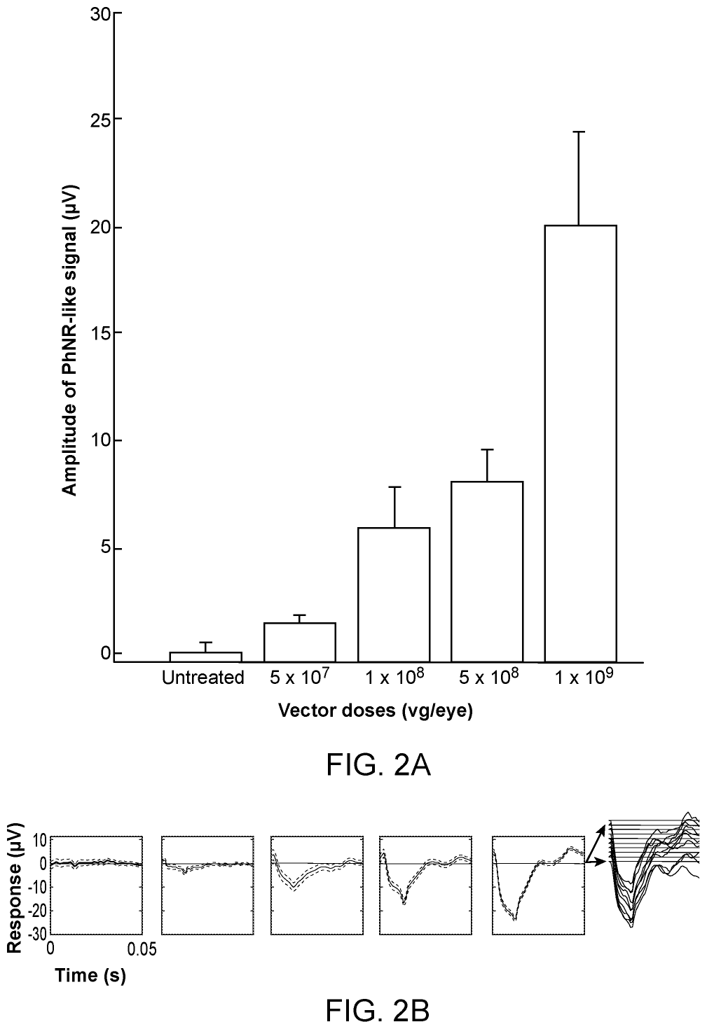

Following the experiments shown in FIGS. 1A-1B, the effectiveness of Compound A over a broader range of doses was explored, from 10-fold lower (5×10⁷ vg/eye) to 2-fold higher (1×10⁹ vg/eye). FIGS. 2A-2B shows the results over this 20-fold range; all dose levels were statistically significantly different from the control group (p<0.01, Student's t-test, comparing each dose group to the untreated group), and, the amplitudes of the responses increased with increasing vector dose.

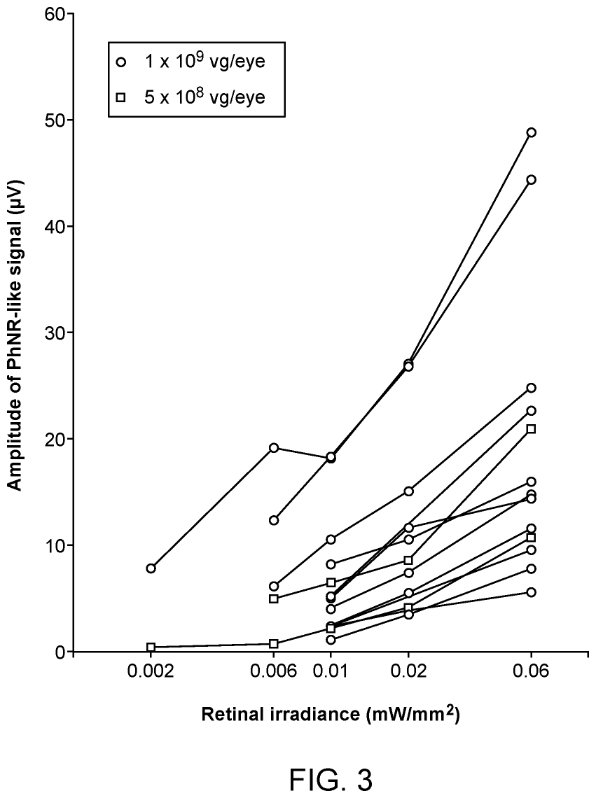

Given the robust PhNR-like responses at the higher vector doses, the intensity of the light stimulation was also tested to consider whether it could be reduced and still produce a light response. Specifically, using the two highest doses, consideration of whether the light level could be dropped while still maintaining a PhNR-like amplitude that was significantly above the control level. The results showed that the light level required for a vector dose of 1×10⁹ vg/eye could be reduced by approximately a factor of 10 (FIG. 3). For the next highest dose, 5×10⁸ vg/eye, the light level could be reduced by a factor of 6 (p<0.01, Student's t-test). The effects on lower vector doses were not tested, since there was little room for adjustment. These results served to narrow the range of light levels needed for testing in a clinical trial, reducing the burden of exploration with patients. The controls in this study (FIGS. 2A-2B), which were blind, untreated eyes, always received the maximal light intensity (0.06 mW/mm²). Thus, without the vector treatment, blind eyes showed no response even to the brightest light in this regimen, whereas with Compound A treatment, the eyes responded with robust ERG signals even when light levels were dropped substantially. Light levels may be dropped further when perceptual studies are performed with human subjects, since ERG responses are well known to be less sensitive than perceptual ones.

### Assessing Safety

The ERG responses in rd1 mice demonstrated that Compound A is effective in producing light responses in blind animals. To assess the vector's safety, studies were performed in rats, nonhuman primates, and mice, using several tests, including immunohistochemical analyses and cell counts (rats), electroretinograms (nonhuman primates), and ocular tolerance/toxicology assays (mice).

### Assessing Safety Using Immunohistochemical Analyses and Cell Counts in Rats

Similar to the rd1 mouse line and human patients with RP, S334ter-3 rats have an inherited retinal degenerative disease that leads to severe loss of photoreceptors (Martinez-Navarrete et al., 2011). Two sets of experiments were performed. The first assessed the safety of the Compound A vector, the second assessed the safety of the Compound A vector combined with the light stimulation needed to activate it.

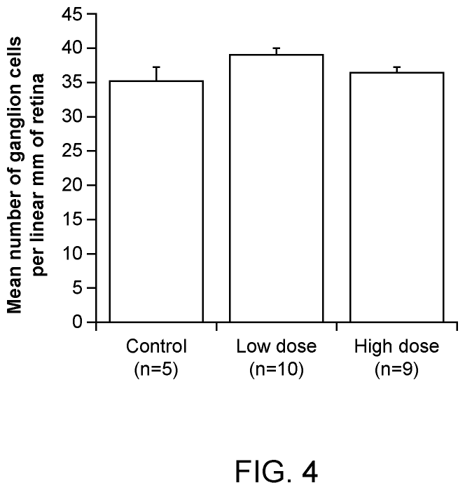

For the first set, 3 groups of animals were used: a low dose group that received 6.8×10⁸ vg/eye, which corresponds to 6.7×10⁷ vg/eye in mouse (Onodera et al., 2015), a high dose group that received 2.7×10⁹ vg/eye, which corresponds to 2.6×10⁸ in mouse, and a control group that contained both vehicle-treated eyes and untreated eyes. Note that there is a wide range of vitreous volumes that have been reported for rat eyes (13-54 μL) (Onodera et al., 2015). The largest volume for the rat vitreous (54 μL) was assumed when converting to the mouse equivalent dose (mouse vitreous is 5.3 μL). Thus, the doses used here may be considerably higher (as much as 4 times higher) and, therefore, provide an even stronger assessment of the toxicity of Compound A to ganglion cells. The assay performed was to count the number of cells in the ganglion cell layer. If treatment with Compound A were detrimental to the targeted cells, one would expect a loss of cells in the ganglion cell layer of the treated retinas compared to controls. Ganglion cell were labeled with two markers, one that labels ganglion cells in general (Brn3) (Nadal-Nicolas et al., 2009) and one that labels the Compound A-expressing cells specifically (an antibody to GFP). Cell counts were performed 5 months after Compound A injection to allow for cell death, if it occurred, as well as removal of cellular debris (Elmore, 2007). FIG. 4 shows the results: there was no statistically significant difference in retinal ganglion cell counts between the low dose group and the control group 5 months after treatment (p>0.1, Student's t-test) or between the high dose group and the control group (p>0.5, Student's t-test)).

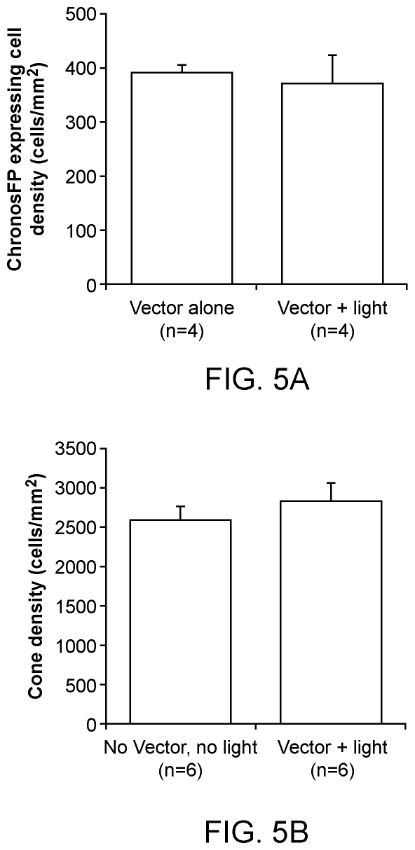

The second set of experiments assessed the safety of the vector plus light stimulation. These were divided into two parts. The first focused on testing for the presence of Compound A-expressing cells. Since these cells had been made light-sensitive by expressing Compound A in them, there was a possibility that light stimulation would damage them, limiting the value of a therapy that required light stimulation. To assess the safety of light-activating Compound A expressing cells, two groups of 4 animals were treated with Compound A, both at 2.7×10⁹ vg/eye. The animals in one group received light stimulation similar to the exposure expected in a clinical trial with an optogenetic vector (12 2-hour sessions over a period of 8 weeks at 0.1 mW/mm², while the other group of animals received no light stimulation. The retinas were processed 5 months after the light stimulation. The results showed no difference in the number of Compound A-expressing cells in the two groups, indicating no loss of Compound A-expressing cells as a result of the light exposure (FIG. 5A) (p>0.7, Student's t-test).

The second part of the study focused on photoreceptors, assessing whether the light stimulation needed to drive Compound A in the ganglion cells caused damage to the naturally light-absorbing cells in the retina, the photoreceptors. For these experiments, wildtype (WT), rather than S334ter-3, animals, were used since adult S334ter-3 rats no longer have a photoreceptor layer as a result of their retinal degenerative disease (Martinez-Navarrete et al., 2011; McGill et al., 2012). Six WT animals were assessed. In each WT animal, one eye received Compound A plus light treatment (12 2.5-hour sessions over a period of 6 weeks at 0.1 mW/mm²), and the other eye received no treatment. The Compound A dose was 8.4×10⁹ vg/eye, which corresponds to 8.2×10⁸ vg/eye in mouse. The retinas were removed 6 months post-injection. The results (FIG. 5B) showed no loss of photoreceptors (e.g., cones) in the Compound A-plus light treated retinas compared to the untreated retinas (p>0.4, Student's t-test). These results are consistent with previous results on light stimulation for optogenetic therapies reported in Yan et al. (2016) showing no loss to the photoreceptor layer, which contains both rods and cones, as measured by retinal thickness (outer nuclear layer thickness) and using the same wavelength and light level.

### Accessing the Safety of Compound A in Nonhuman Primates (NHPs) Using Electroretinograms

The study in rats assessed tissue integrity using immunohistochemistry and cell count assays. To examine electrophysiological safety and in a species similar to humans, ERGs were performed on cynomolgus macaques. Three animals (6 eyes total) were assessed 7 months after vector administration by intravitreal injection. One animal (both eyes) received Compound A and two animals (both eyes) received a variant that used the same transgene (SEQ ID NO: 2) but was packaged using a different AAV2 capsid (the AAV2tYF capsid (Petrs-Silva et al., 2009). The doses fell within the ranged used in the efficacy study in FIGS. 2A-2B; doses were 3.7×10¹⁰ vg/eye and 1.2×10¹¹ vg/eye, which is equivalent to 1×10⁸ vg/eye and 3.2×10⁸ vg/eye in mouse (Onodera et al., 2015). Four animals (8 eyes total) were untreated and served as controls.

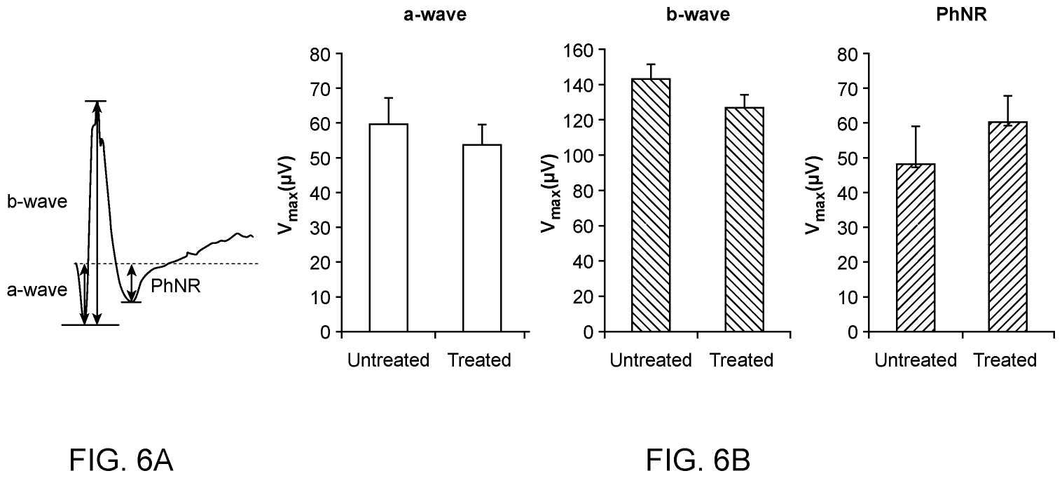

Whether ERG responses were adversely affected in treated versus untreated eyes, using the three standard photopic ERG components: the a- and b-waves, which reflect photoreceptor and bipolar cell responses, respectively, and the PhNR wave, which reflects ganglion cell responses. Seven months after vector injection, intensity/response data were fit to a generalized Naka-Rushton function to derive the saturated amplitude for each wave, the Vₘₐₓ, following reference (Joshi, Ly, and Viswanathan, 2017). As shown in FIGS. 6A-6B, there was no statistically significant reduction in Vₘₐₓ observed for any of the three ERG components as a result of the treatment (p>0.2, for all waves, comparing the Vₘₐₓ values in the treated group with those in the untreated group).

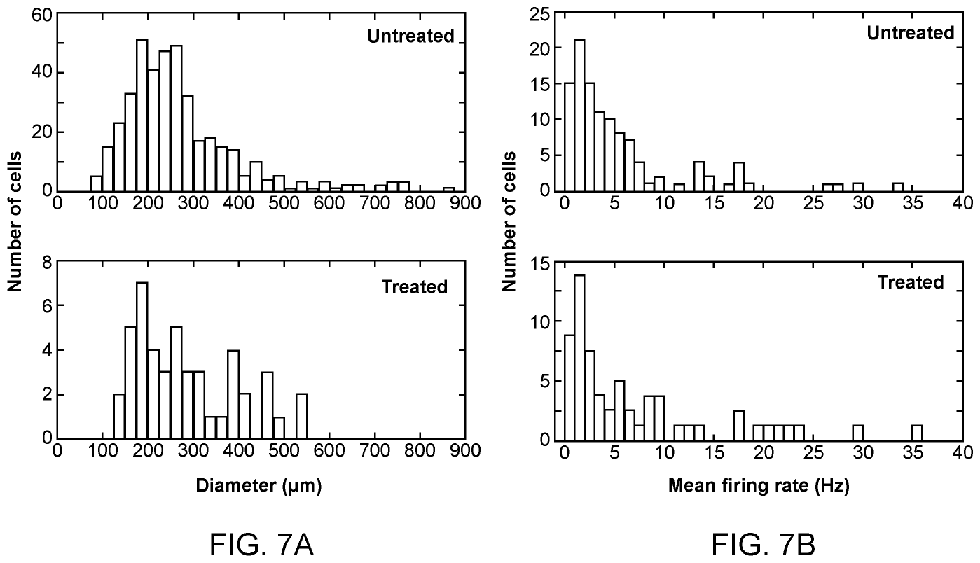

The ERG experiments assess physiological function at a macroscale, e.g., whole retina electrophysiology. Multi-electrode array (MEA) recordings from excised retinas of cynomolgus macaques that were previously injected with SEQ ID NO: 3-expressing vectors were evaluated for receptive field size and mean firing rate. The retinas from 6 eyes (3 animals) were treated with an array of SEQ ID NO: 3 expressing vectors. For these experiments, the capsid was the AAV2 variant AAV2tYF, the promoters were CAMKII, hCACNA1G, and mNefL1.6, and the dose range was from 2.2×10¹¹ to 7.6×10¹² vg/eye (equivalent to 5.8×10⁸ vg/eye to 2×10¹⁰ vg/eye in mouse). The results showed that the distributions of receptive field sizes and firing rates from the SEQ ID NO: 3-treated group were not statistically significantly different from those from the untreated group (FIGS. 7A-7B), (p>0.2, Kolmogorov-Smirnov test). Retinal ganglion cells sampled from eyes that had been injected intravitreally with a SEQ ID NO: 3-expressing vector showed receptive field sizes and firing rates that were very similar to those from untreated retinas, with the stimuli used to assess firing rates drawn from natural scenes (e.g., trees, landscapes, people walking).

### Assessing Local Tolerance in Blind Mice

The safety of Compound A in terms of local tolerance was assessed. A total of 120 rd1 mice were divided into three groups (40 animals per group): two dose groups spanning a factor of 10 in dose level (4.25×10⁸ vg/eye and 4.25×10⁹ vg/eye) and a vehicle-alone group. Injections were performed intravitreally to the right eye, and the left eye remained untreated. Each group had two sacrifice time points, Week 4 and Week 12 (20 animals at each time point).

SEQ ID NO: 3 expression in the injected eyes was verified. SEQ ID NO: 3 expression, measured by GFP-immunolabeling (magenta), was detected in nerve fiber layer, inner plexiform layer, optic disc and the extending axons in all animals.

Ophthalmic examinations were performed on week 2, 4, 8 and 12 post-injection, and the findings are summarized in Table 1, below. A slit lamp was used to assess anterior segments including cornea, iris, and lens. An indirect ophthalmoscope was used to assess the posterior segments including vitreous chambers and retinas. Previous phenotypic characterizations of rd1 mice have shown that their eyes have vessel attenuation and pigment patches at an early age (Hawes et al., 1999; Chang et al., 2002). Consistent with this, these phenotypes were present at similar frequencies in both the Compound A and vehicle-injected eyes in our study, suggesting that they were not caused by the vector.

In hematoxylin and eosin-stained retinal sections, Compound A-related microscopic findings included minimal to slight mononuclear cell infiltrates in the vitreous (Timmers et al., 2020), where minimal is the lowest level in a 5 level classification (Table 2, below). By terminal sacrifice (Week 12), only the lowest (minimal level) mononuclear cell infiltrates were present.

TABLE 1
Ophthalmic examination findings of injected eyes

| | | | | | Group 2 | | | | Group 3 | | | |
|---|---|---|---|---|---|---|---|---|---|---|---|---|
| | Group 1 | | | | 4.25 × 10⁸ vg/eye | | | | 4.25 × 10⁹ vg/eye | | | |
| | Vehicle | | | | (Low Dose) | | | | (High Dose) | | | |
| Study Week | 2 | 4 | 8 | 12 | 2 | 4 | 8 | 12 | 2 | 4 | 8 | 12 |
| Corneal | 6/40 | 8/40 | 1/20 | 2/20 | 4/40 | 3/40 | 2/20 | 4/20 | 10/40 | 9/40 | 4/20 | 7/20 |
| Opacity | | | | | | | | | | | | |
| Cataract | 8/40 | 9/40 | 3/20 | 2/20 | 4/40 | 2/40 | 6/20 | 2/20 | 6/40 | 3/40 | 2/20 | 0/20 |
| Vitreal | 24/40 | 19/40 | 8/20 | 4/20 | 17/40 | 9/40 | 7/20 | 1/20 | 27/40 | 25/40 | 17/20 | 9/20 |
| Degeneration | | | | | | | | | | | | |
| Pigment | 40/40 | 40/40 | 20/20 | 20/20 | 40/40 | 40/40 | 20/20 | 20/20 | 40/40 | 40/40 | 20/20 | 20/20 |
| Clumping | | | | | | | | | | | | |
| Retinal | 39/40 | 40/40 | 20/20 | 20/20 | 40/40 | 40/40 | 20/20 | 20/20 | 40/40 | 40/40 | 20/20 | 20/20 |
| Vessel | | | | | | | | | | | | |
| Attenuation | | | | | | | | | | | | |
| Vitreal | 1/40 | 0/40 | 0/20 | 0/20 | 0/40 | 0/40 | 0/20 | 0/20 | 1/40 | 1/40 | 0/20 | 0/20 |
| Hemorrhage | | | | | | | | | | | | |

TABLE 2¹
Incidence and severity of test article-related microscopic findings: mononuclear
cell infiltration in the vitreous reverted to lowest level (minimal) on Week 12

| | Interim Sacrifice (Week 4) | | | | | | Terminal Sacrifice (Week 12) | | | | | |
|---|---|---|---|---|---|---|---|---|---|---|---|---|
| | | | Group 2 | | Group 3 | | | | Group 2 | | Group 3 | |
| | | | 4.25 × 10⁸ | | 4.25 × 10⁹ | | | | 4.25 × 10⁸ | | 4.25 × 10⁹ | |
| | Group 1 | | vg/eye | | vg/eye | | Group 1 | | vg/eye | | vg/eye | |
| | Vehicle | | (Low Dose) | | (High Dose) | | Vehicle | | (Low Dose) | | (High Dose) | |
| Sex | M | F | M | F | M | F | M | F | M | F | M | F |
| Minimal | 0/5 | 0/5 | 0/5 | 1/5 | 3/5 | 3/5 | 0/5 | 0/5 | 1/5 | 0/5 | 3/5 | 3/5 |
| Slight | 0/5 | 0/5 | 0/5 | 0/5 | 1/5 | 1/5 | 0/5 | 0/5 | 0/5 | 0/5 | 0/5 | 0/5 |

¹At each sacrifice, only half of the animals per group (10 animals per group) were processed for pathology examination; the remaining 10 were kept for a potential biodistribution study in the future, if needed.

### Example 2: A Single-Site, Phase 1/2, Safety and Efficacy Trial of Compound A, a Recombinant Adeno-Associated Virus Vector Having SEQ ID NO: 1 in Patients with Retinitis Pigmentosa

### Study Design

A non-randomized, open-label, Phase 1/2 dose escalation study of the safety and efficacy of Compound A is being conducted by administering Compound A, Compound A, utilizing SEQ ID NO: 3 as the optogenetic fusion protein by intravitreal injection in one eye in individuals with retinitis pigmentosa (RP). Secondary study outcomes and efficacy are noted below.

Each participant receives Compound A by intravitreal injection in one eye on a single occasion as outlined in Table 3, below.

TABLE 3
Design of Clinical Trial

| | | | | Vector dose | | |
|---|---|---|---|---|---|---|
| | | | | Concen- | | |
| | | Age | Route of | tration | Volume | Total dose |
| Group | N | (yr) | delivery | (vg/mL) | (μL) | (vg/eye) |
| 1 | 3^{a} | ≥18 | Intravitreal | 1 × 10¹² | 100 | 1 × 10¹¹ |
| 2 | 3^{a} | ≥18 | Intravitreal | 3.0 × 10¹² | 100 | 3.0 × 10¹¹ |
| 3 | 3^{a} | ≥18 | Intravitreal | 6.0 × 10¹² | 100 | 6.0 × 10¹¹ |
| 4 | 3^{a} | ≥18 | Intravitreal | 1.2 × 10¹³ | 100 | 1.2 × 10¹² |
| 5 | 28 | ≥18 | Intravitreal | MTD^{b} | 100 | MTD^{b} |

^{a}Approximately 3 per group, not to exceed 5
^{b}MTD = maximum tolerated dose, i.e., the dose below which a dose limiting toxicity (DLT) is assessed by the principal investigator. If no DLT is observed, then the MTD will be the highest dose tested.

Participants in all groups are least 18 years of age and receive the vector at a low dose (Group 1), a low-middle dose (Group 2), a high-middle dose (Group 3), or a high dose (Group 4). Participants in Group 5 are at least 18 years of age and receive the vector at the maximum tolerated dose (MTD) determined in Groups 1, 2, 3 and 4.

When the safety evaluations identify no dose-limiting toxicity in Group 1, subjects will be enrolled in Group 2. When the safety evaluations identify no dose-limiting toxicity in Groups 1 and 2, participants will be enrolled in Group 3. The MTD will be the dose below which a dose-limiting toxicity (DLT) is observed by the principal investigator. If no DLT is observed, then the MTD will be the highest dose tested. After review of safety data from Groups 1, 2, 3 and 4 by the DSMC, participants may be enrolled in the dose expansion group, Group 5, at the MTD identified in previous groups. If the DSMC determines from the safety data at Day 14 of any group that the MTD has been met, no further dose escalation will occur, and all remaining subjects will be assigned to the MTD group.

Within Groups 1, 2, 3, and 4, enrollment of participants are staggered by at least 2 weeks to allow adequate time for review of safety information by the investigators and sponsor.

Safety is monitored by evaluation of ocular and non-ocular adverse events/adverse experiences (AEs), and hematology and clinical chemistry parameters. Other data collected will be immune responses to SEQ ID NO: 3 and presence of vector DNA in the blood, saliva and tears.

Efficacy is measured by evaluation of light threshold and motion and shape/object detection (see Specification of Efficacy Parameters section, below).

Other data collected may include electrophysiological measures (ERG and/or VEP), fundus photography, immune responses to AAV and quality of life questionnaires.

Up to approximately 40 participants will be enrolled in this study. Enrollment in this study is anticipated to take approximately 30 months. Enrolled participants will have frequent follow-up visits during the first year after study agent administration.

To monitor for delayed AEs and assess the duration of any changes in visual function or structure that occur, participants will be following annually for an additional 4 years after the Month 12 visit.

### Specification of Efficacy Parameters

The following lists the efficacy parameters. Procedures for obtaining them are provided below in Visual Function Measurements section, below.

Changes in light detection (threshold to detect) in the treated eye will be measured using a staircase or binary search procedure.

Changes in motion detection will be measured using a standard two-alternative forced choice paradigm.

Changes in shape detection will be measured using a standard two-alternative forced choice paradigm.

Changes in amplitude of Visual Evoked Potential (VEP) or ERG signals will be measured.

### Visual Function Measurements

### Psychophysical (Perceptual) Measurements

Psychophysical measurements in the study eye will draw from the standard battery of tests used with subjects with ultra-low vision: light detection, motion detection, and shape/object detection (Bach et al., 2010; Wilke et al., 2007).

Primary efficacy endpoint: the primary efficacy endpoint is light detection (e.g., changes in threshold to detect). This will be assessed using a stimulator (Diagnosys LLC) that present flashes of light at different intensities following a staircase or binary search procedure to determine the subject's threshold response. Subjects will be tested at the baseline visit and in conjunction with the safety assessment visits at approximately 3, 6, 9 and 12 months, and at each long-term follow-up visit. This test assesses whether the optogenetic protein in Compound A is being expressed at a sufficient level to produce perceptual responses in RP subjects. It also assesses the minimum light level needed to activate the protein for the subsequent tests assessing light, motion and shape detection.

Secondary efficacy endpoints: secondary endpoints will be assessed using a hierarchy of tests, based on difficulty. They include motion detection, detection of the direction of motion and shape/object recognition, and they will be performed using a PSIII pattern stimulator (Diagnosys LLC) or an easier to use pattern stimulator (Optecks), which is a portable testing device that provides a wider field of vision. Test results of the two stimulators will be assessed and documented.

The tests will be conducted using standard forced-choice paradigms under conditions where the subjects provide their responses in a self-paced manner using a keypad or button box. With respect to advancing through the hierarchy, if a subject's performance on a given test is at chance, the investigator or assessor can choose not to advance to the next level of difficulty, so as not to burden the subject unnecessarily on tests he or she is unable to perform (e.g., if he/she cannot detect motion vs no motion in ˜10 presentations, then tests that build on this, such as detection of leftward vs. rightward motion may be excluded in the testing series).

Motion detection (e.g., direction of motion): Stimuli are presented in a two-alternative forced choice paradigm. Briefly, on each trial, the subject is presented with a bar moving in one of two directions in the visual field, chosen randomly and with equal probability, and he/she must indicate the direction of motion. Performance is measured as the fraction correct over all trials. A 0.5 fraction correct is chance, and values above this will be assessed using standard statistics. If subject is able to perform well, a 4-alternative choice test may be performed.

Shape/object detection: Stimuli are presented in a two-alternative forced choice paradigm. Briefly, on each trial, the subject is presented with one of two shapes (e.g., a target shape or a non-target shape), chosen randomly and with equal probability, and he/she must indicate whether or not the presented stimulus is the target shape. Performance is measured as the fraction correct over all trials. A 0.5 fraction correct is chance, and values above this will be assessed using standard statistics. If subject is able to perform well, a 4-alternative choice test may be performed.

Additional non-invasive measures of visual function with or without the stimulators, such as object detection (e.g., household objects, items in the refrigerator) or light localization may also be performed at the discretion of the investigator or assessor.

Training visits will also be conducted. Between baseline and Day 0 a familiarization visit will be conducted with the subject to orient him or her to the stimulation devices and testing procedures and to record baseline test values. In addition, subsequent to study agent administration, training sessions will be scheduled in approximate conjunction with the Month 3-6 visits to allow optimization of the stimulus to the subject's foveal ring, and to allow the subject to develop familiarity with the new input, in particular to develop an association between the optogenetic activation of his or her retinal ganglion cells and visual perceptions. The number of training sessions and the duration of the testing time is at the discretion of the investigator; sessions are expected to last approximately two hours.

### Results

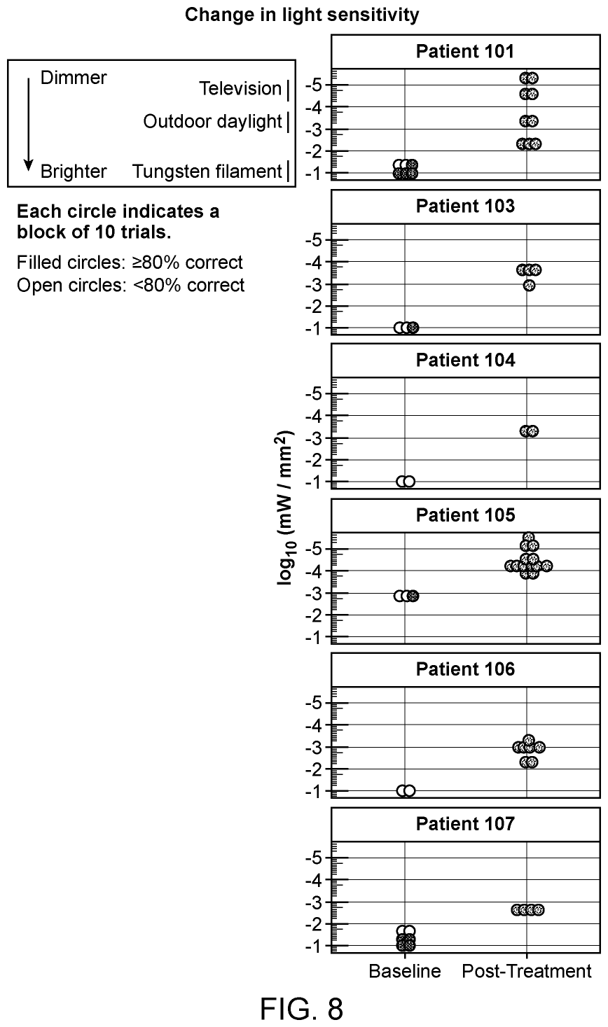

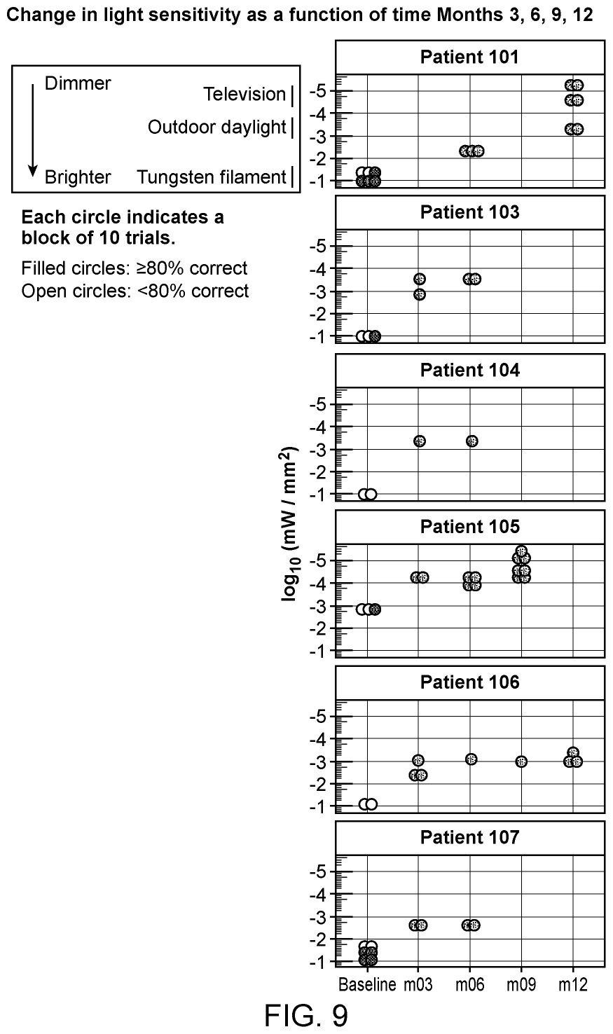

In the 2-alternative forced choice task, on each trial, the patient pressed a bar to trigger the stimulus: either light (3 flashes) or no light (for an equal amount of time as the 3 flashes). An automated voice would then ask the patient to make a choice, and he/she would press a button to indicate the answer. Stimuli were delivered automatically and randomly interleaved. FIG. 8 shows that all patients showed an increase in light sensitivity. Note that the y-axis is on the log scale, indicating that the increase in light sensitivity was substantial. These data provide evidence that Compound A was taken up and expressed SEQ ID NO: 3 sufficiently to produce a light response. FIG. 9 shows that this improvement in light sensitivity was stable or increased throughout the period of time tested (up to 12 months) for all patients. These early durability data suggests that benefits of Compound A are sustained.

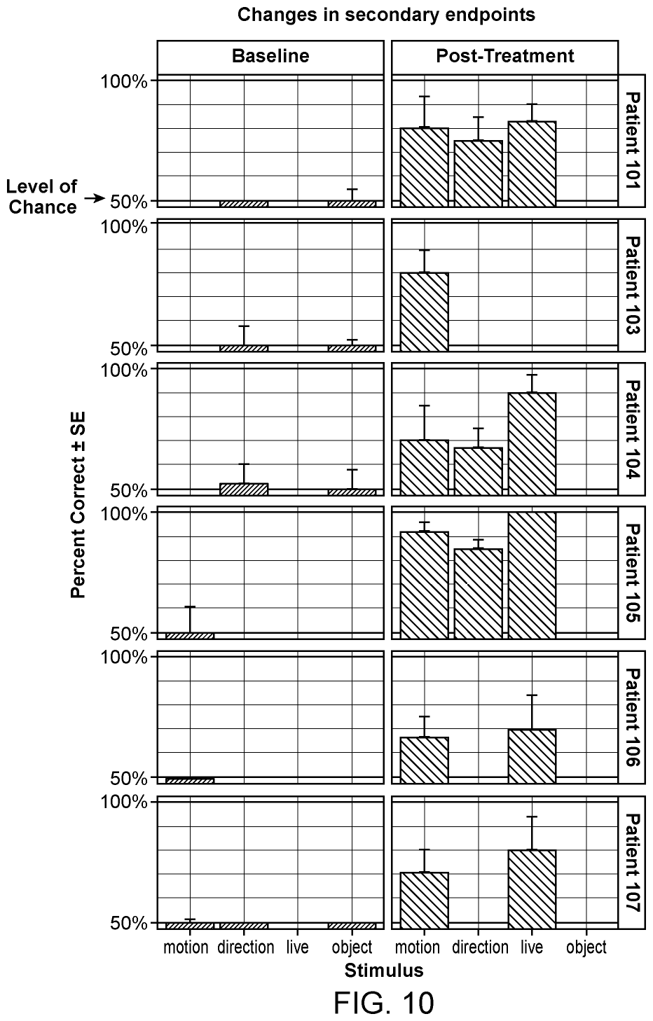

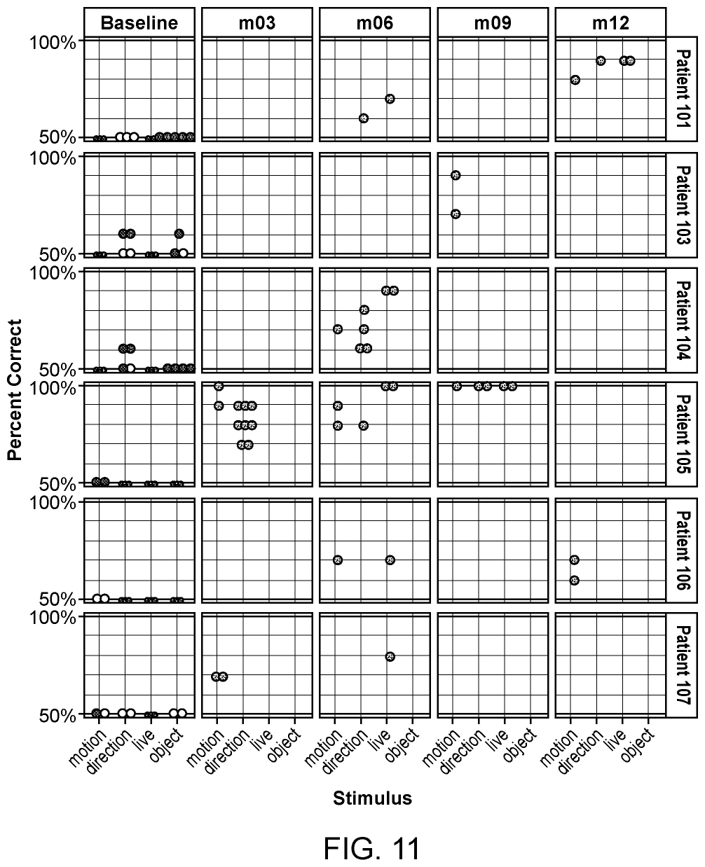

Using the motion detection and shape/object paradigms described above, four endpoints were assessed (ranked in a hierarchy of difficulty), including the ability to detect motion, the ability to detect the direction of motion, the ability to distinguish live actions (e.g., arms moving up vs. down or e.g., an arm moving vs. a hand moving), and the ability to distinguish objects (e.g., an apple vs. other fruits or vegetables or e.g., the suits on playing cards). It was observed that patients showed improvements on several secondary endpoints. In particular, at baseline performance was at chance or lower for all the tasks (chance is at 50% for a 2-alternative task). FIG. 10 shows that after treatment, all patients gained the ability to detect motion and several patients also gained the ability to detect the direction of motion and live action. No patients at the lower vector doses were able to recognize objects. FIG. 11 show that the improvements remained stable or actually increased over the period of time tested (up to 12 months).

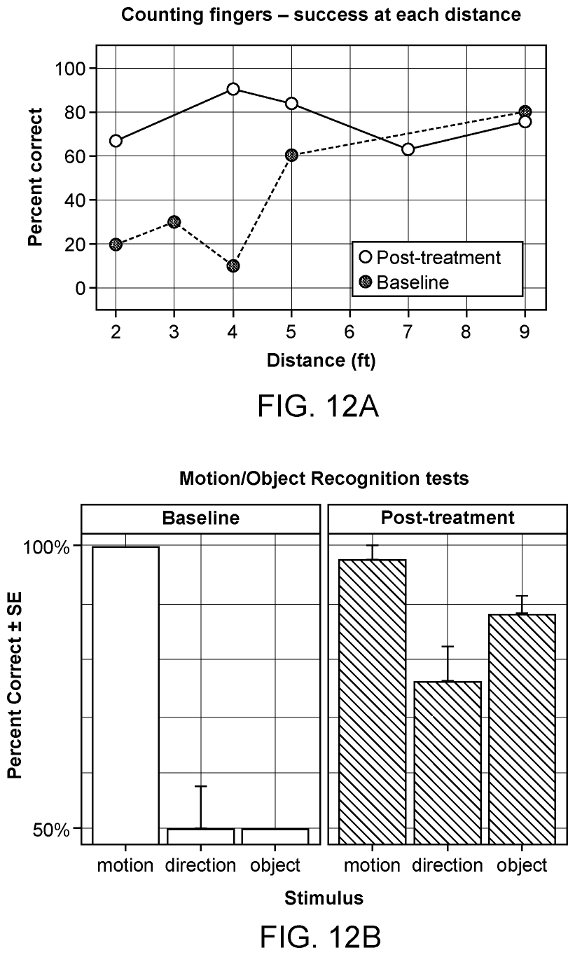

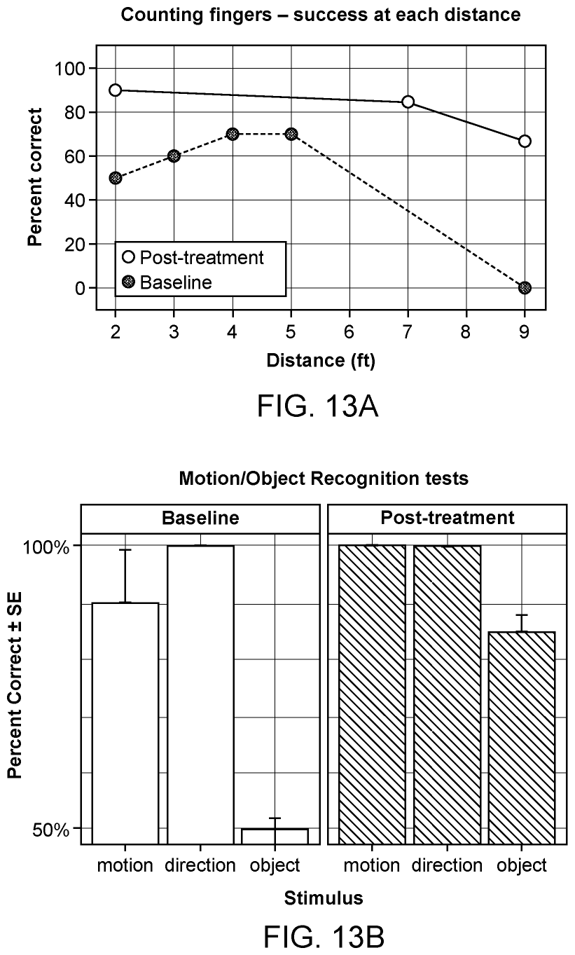

Patients in a finger counting task were tested (FIGS. 12A and 13A), which entails detection motion, detecting the direction of motion, and recognizing objects; as well as a set of motion/object recognition tests at baseline or post treatment (FIGS. 12B and 13B). The two depicted patients had a baseline visual acuity: <20/200 and received the Compound A dose of 6.0×10¹¹ vg/eye (Cohort 3). Finger count testing was performed at various distances from the patient. At each distance, a block of 8-10 trials was presented, randomly interleaved, and percent correct at each distance was tabulated. All other testing was performed as previously described. Baseline tests were 2-alternative choice tasks. Post-treatment tests were either 2-alternative or, more challengingly, 4-alternative choice tasks. It was observed that both finger counting and motion/object recognition improved in both patients (FIGS. 12A-B and FIGS. 13A-13B). Note that at baseline, this patient could detect motion, but not the direction of motion, nor the objects on the object recognition task. After treatment, the patient was able to detect the direction of motion and readily perform the object recognition task, often calling out the objects as she saw them (fruits, vegetables, suits on playing cards).

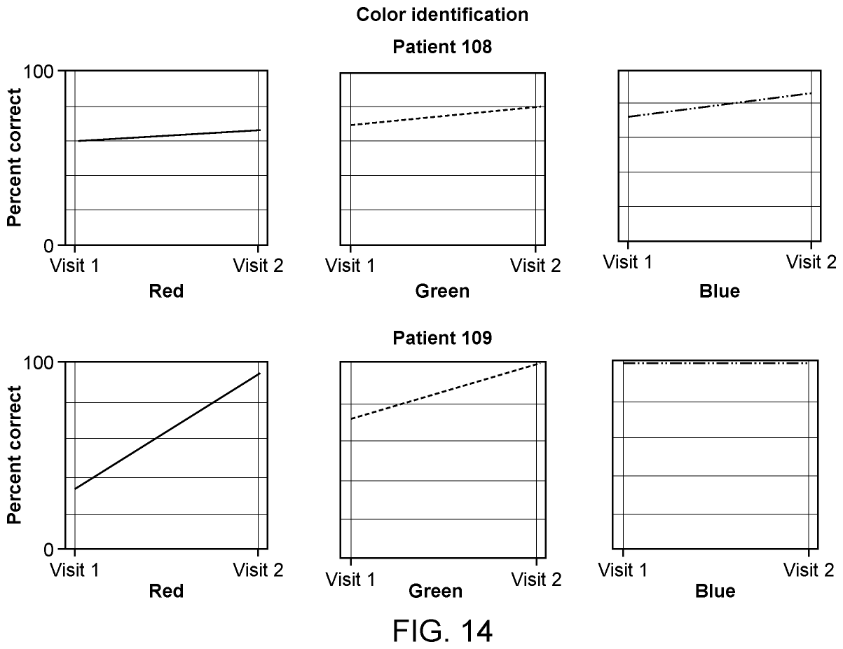

Patients were additionally tested in a color identification task. The two depicted patients had a baseline visual acuity: <20/200 and received the Compound A dose of 6.0×10¹¹ vg/eye (Cohort 3). On each trial, the patient was presented with a shape that was either red, green or blue. Percent correct for each color was tabulated. These tests were performed post-treatment at 2 sets of visits. Patient 108 showed a modest improvement in distinguishing red, green, and blue. Patient 109 showed a striking improvement in her ability to see red, a modest improvement in her ability to see green, and maintained her ability to see blue (FIG. 14). These results were consistent with subject 109's observations about color at home: she suddenly saw that her couch was red and not black, as she had thought, and became newly able to distinguish among her colored pills (red vs blue), improving quality of life.

The results described above provide clinical proof of concept. In particular, patients who started with complete or near-complete blindness can now see light, and their sensitivity increased with time, with several being able to see light at daylight or television levels. All of these patients can now also detect motion, including three who can also detect the direction of motion, both with computer generated images and live action. Early data from patients with higher levels of residual retinal function suggest potential for greater functional recovery.

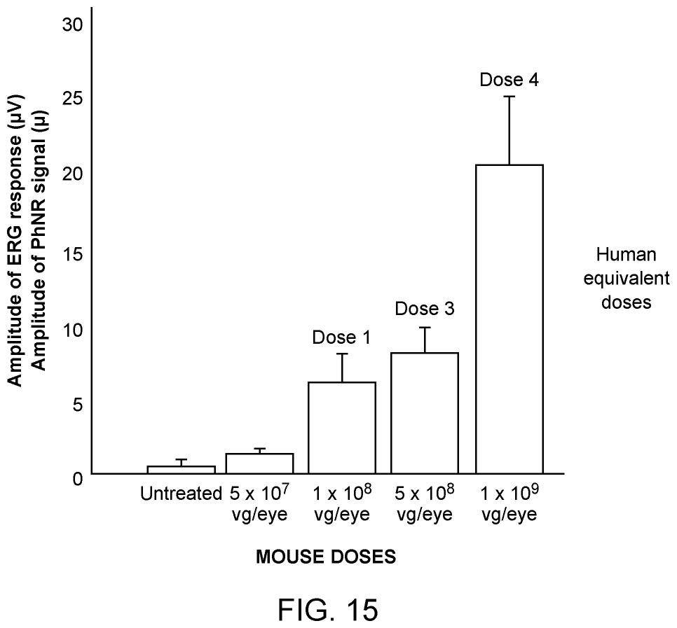

Additional studies address whether Compound A dose escalation can be used to achieve incremental benefits. These studies are based upon in vivo data from mice demonstrating that there is a non-linear dose response observed in preclinical species (FIG. 15).

### Example 3: Design of Vectors Containing a Gene that Expresses an Optogenetic Protein

A gene encoding an optogenetic fusion protein (e.g., a protein having the amino acid of SEQ ID NO: 3) can be prepared, for instance, by procedures known in the art. Techniques for solid phase synthesis of polynucleotides are known in the art and are described, for instance, in U.S. Pat. No. 5,541,307, the disclosure of which is incorporated herein by reference as it pertains to solid phase polynucleotide synthesis and purification. Additionally, the prepared gene can be amplified, for instance, using polymerase chain reaction (PCR)-based techniques known in the art and/or by transformation of E. coli with a plasmid containing the optogenetic fusion protein. The bacteria can subsequently be cultured so as to amplify the DNA therein, and the gene encoding the optogenetic fusion protein can be isolated by plasmid purification techniques, known in the art, followed by a restriction digest and/or sequencing of the plasmid to verify the identity of the optogenetic fusion protein.

A DNA polynucleotide encoding an optogenetic fusion protein or an RNA equivalent thereof may exhibit at least 90% (e.g., at least 91%, 92%, 93%, 94%, 95%, 96%, 97%, 98%, or 99%) sequence identity to the nucleic acid sequence of SEQ ID NO: 2. In another example, the DNA polynucleotide encoding an optogenetic fusion protein may have a nucleic acid sequence that is identical to the nucleic acid sequence of SEQ ID NO: 2. The optogenetic fusion protein may have the amino acid sequence of SEQ ID NO: 3

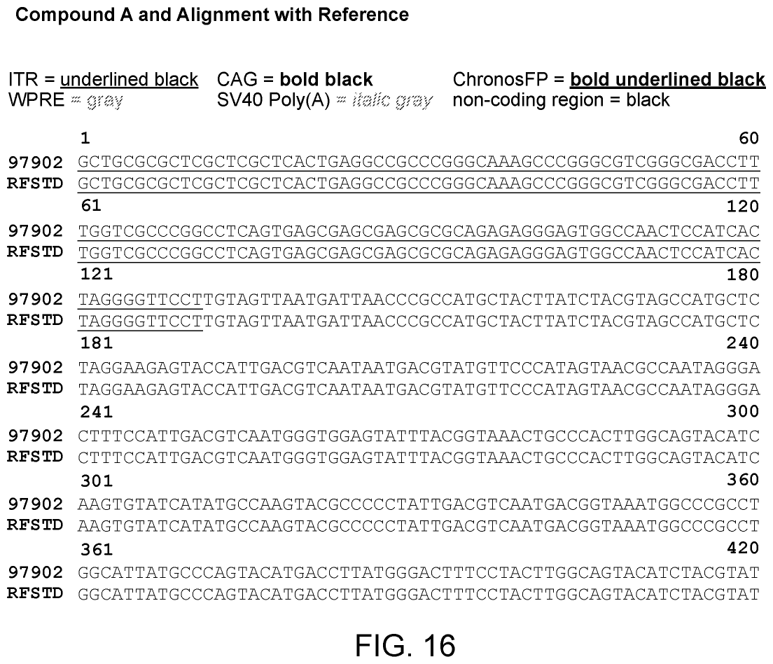

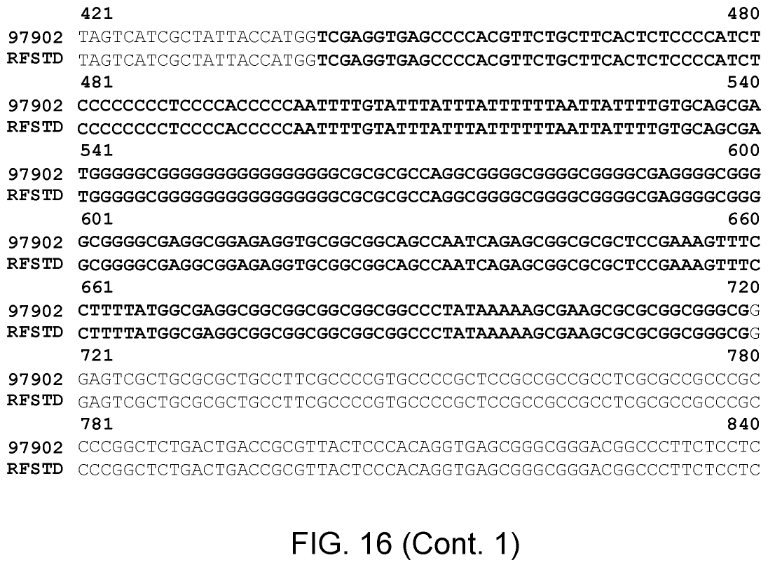

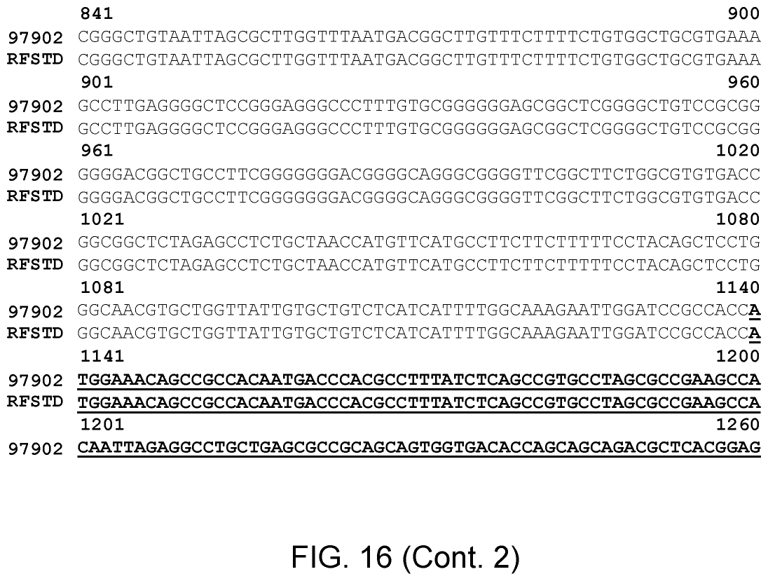

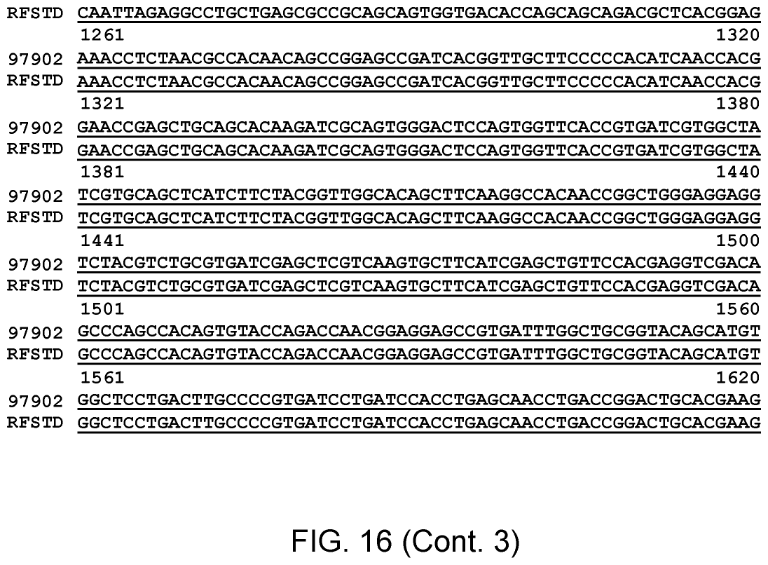

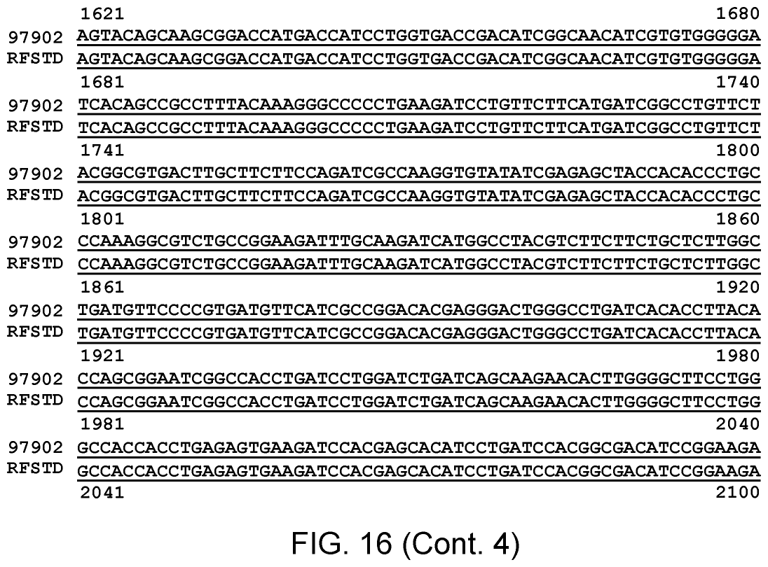

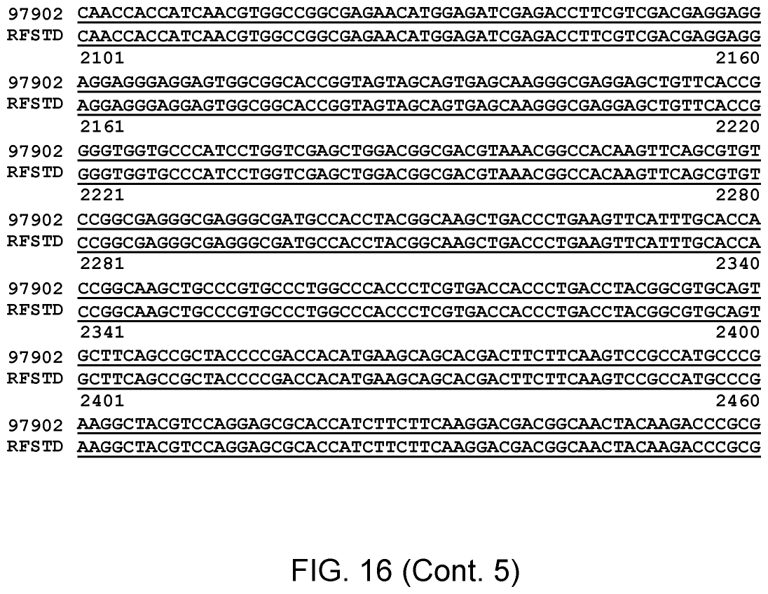

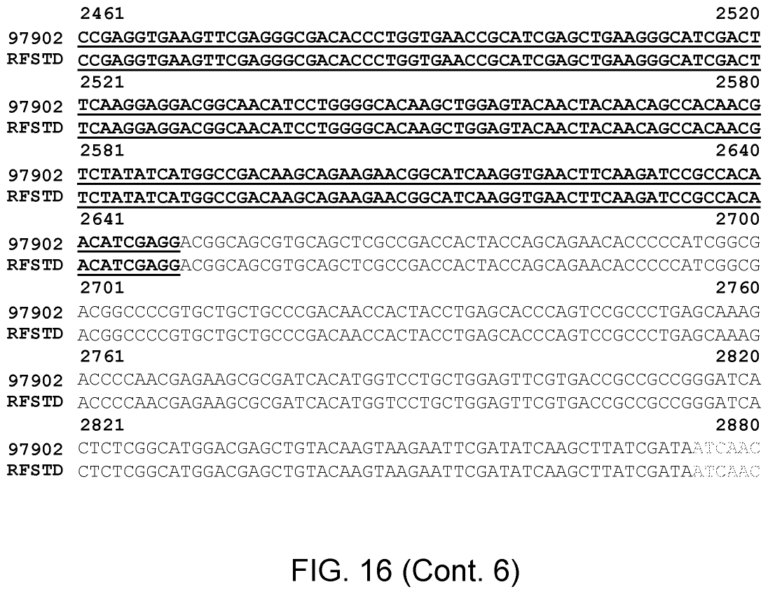

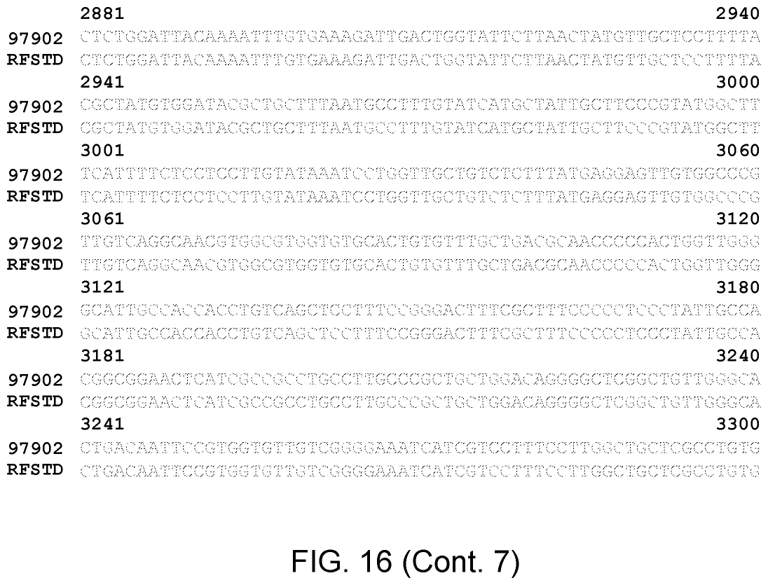

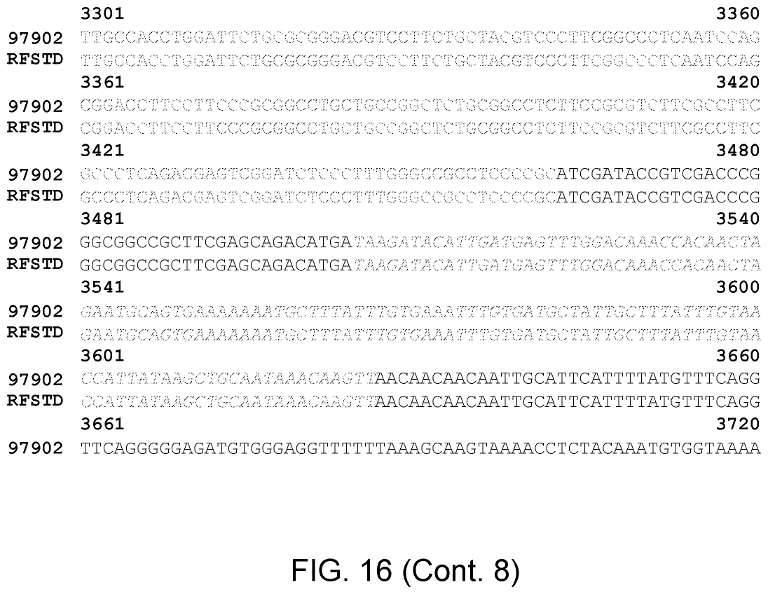

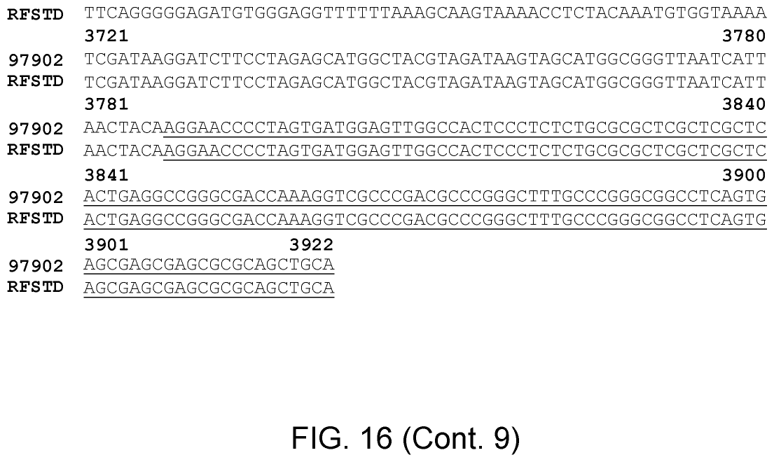

The DNA nucleotide encoding an optogenetic fusion protein or an RNA equivalent thereof can subsequently be incorporated into a plasmid, such as a viral vector. For instance, an adeno-associated virus (AAV) vector, such as an AAV2 can be generated that incorporates the optogenetic fusion protein (e.g., SEQ ID NO: 3) between the 5′ and 3′ inverted terminal repeats of the vector, and the DNA nucleotide may be operably linked to a constitutive promoter (e.g., a CAG promoter). Such a vector may exhibit at least 90% (e.g., at least 91%, 92%, 93%, 94%, 95%, 96%, 97%, 98%, or 99%) sequence identity to the nucleic acid sequence of SEQ ID NO: 1. In another example, the AAV encoding an optogenetic fusion protein may have a nucleic acid sequence that is identical to the nucleic acid sequence of SEQ ID NO: 1 (FIG. 16).

A practitioner of skill in the art can monitor the expression of the optogenetic fusion protein by a variety of methods. For instance, one of skill in the art can transfect cultured cells with a viral vector that includes an optogenetic fusion protein. Expression of the encoded fusion protein can subsequently be monitored using, for example, an expression assay, such as qPCR, RNA Seq, ELISA, or an immunoblot procedure.

### Example 4: Treatment of Retinitis Pigmentosa by Administration of Vectors Containing a Gene that Expresses an Optogenetic Protein

Using conventional molecular biology techniques known in the art, a gene encoding an optogenetic protein, such as SEQ ID NO: 4, can be incorporated into a vector, such as a viral vector, and administered to a patient suffering from retinitis pigmentosa. For instance, a patient suffering from retinitis pigmentosa can be administered a viral vector containing a gene that expresses SEQ ID NO: 4 under the control of a transcriptional regulatory element that promotes SEQ ID NO: 4 expression in retinal ganglion cells. For instance, an AAV vector, such as a AAV2 vector, can be generated that incorporates SEQ ID NO: 4 (e.g., the vector may consist or comprise the nucleic acid sequence of SEQ ID NO: 1) between the 5′ and 3′ inverted terminal repeats of the vector, and the gene may be placed under control of a transcriptional regulatory element. The AAV vector can be administered to the subject by a variety of routes of administration, such as intravitreally, for example, in an initial dose of at least about 1×10¹¹ vg/eye.

Following administration of the vector to a patient, a practitioner of skill in the art can monitor the patient's improvement in response to the therapy, by a variety of methods. For instance, a physician can monitor the patient's improvement in light sensitivity, ability to detect motion, the ability to detect the direction of motion, ability to distinguish live actions (e.g., arms moving up vs. down or e.g., an arm moving vs. a hand moving), ability to distinguish objects (e.g., an apple vs. other fruits or vegetables or e.g., the suits on playing cards), ability to count fingers, and/or ability to identify colors. A finding that the patient's function has improved in one of the exemplary tests described above following administration of the therapy may indicate that the patient is responding favorably to the treatment. Subsequent doses can be determined and administered as needed.

### INCORPORATION BY REFERENCE

The entire disclosure of each of the patent documents and scientific articles cited herein is incorporated by reference for all purposes.

### EQUIVALENTS

The disclosure can be embodied in other specific forms without departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. A method of treating retinal degenerative disorder in a human patient in need thereof, comprising administering intravitreally to the patient's eye an initial vector dose of an effective amount of an AAV2 vector having a gene that expresses an optogenetic protein, wherein the vector comprises a nucleic acid sequence that has at least 95% sequence identity to the nucleic acid sequence of SEQ ID NO: 9.

2. The method of claim 1, wherein the vector comprises the nucleic acid sequence of SEQ ID NO: 9.

3. The method of claim 2, wherein the vector comprises a nucleic acid sequence that has at least 95% sequence identity to the nucleic acid sequence of SEQ ID NO: 1.

4. The method of claim 3, wherein the vector comprises the nucleic acid sequence of SEQ ID NO: 1.

5. The method of claim 1, further comprising exposing the patient to light after administering the initial dose, whereby the light is ambient light or light stimulation via a device.

6. The method of claim 1, further comprising administering neurally-coded light stimulation after administering the initial dose.

7. The method of claim 6, wherein the neurally-coded light stimulation is administered at about 5×10⁻² mW/mm² to about 0.1 mW/mm², about 1×10⁻² mW/mm² to about 0.1 mW/mm², about 1×10⁻³ mW/mm² to about 0.1 mW/mm², about 1×10⁻⁴ mW/mm² to about 0.1 mW/mm², or about 1×10⁻⁵ mW/mm² to about 0.1 mW/mm².

8. The method of claim 7, wherein the neurally-coded light stimulation is administered using a neural coding device delivering optogenetic stimulation substantially specific to On-type ganglion cells.

9. The method of any one of claims 1-8, wherein the patient has advanced stage blindness due to the retinal degenerative disorder.

10. The method of any one of claims 1-8, wherein the patient has mild, moderate or severe visual impairment.

11. The method of any one of claims 1-8, wherein the patient has bare light perception (BLP) or no light perception (NLP) at baseline.

12. The method of claim 11, wherein the patient has visual acuity to count fingers or less than visual acuity to count fingers at baseline.

13. The method of any one of claims 1-12, wherein the patient has visual acuity no better than 20/200 or about 20/200 at baseline.

14. The method of any one of claims 1-12, wherein the patient has early stage disease with visual acuity no better than or about 20/60 at baseline.

15. The method of claim 6, wherein the neurally-coded stimulation comprises using a light delivery device that sends light pulses in the retina's neural code, causing the ganglion cell firing to mimic that of the normal retina.

16. The method of any one of claims 1-15, wherein the effective amount is an initial vector dose of about 1×10¹¹ to 1×10¹³ vector genome (vg)/eye.

17. The method of any one of claims 1-15, wherein the effective amount is an initial vector dose of at least about 1.2×10¹² vg/eye.

18. The method of any one of claims 1-15, wherein the initial vector dose is from about 70 μL to about 130 μL with a concentration of about 1×10¹² to about 1×10¹⁴ vg/mL.

19. The method of any one of claims 1-15, wherein the initial vector dose is one of: about 1×10¹¹ vg/eye, about 3.0×10¹¹ vg/eye, about 6.0×10¹¹ vg/eye, about 1.2×10¹² vg/eye or more.

20. A method of improving light sensitivity in a human patient suffering from retinal degenerative disorder, comprising administering intravitreally to the patient's eye an initial dose of at least about 1×10¹¹ vg/eye of an AAV2 vector having a gene that expresses an optogenetic protein, wherein the vector comprises a nucleic acid sequence that has at least 95% sequence identity to the nucleic acid sequence of SEQ ID NO: 9.

21. The method of claim 20, wherein the vector comprises the nucleic acid sequence of SEQ ID NO: 9.

22. The method of claim 21, wherein the vector comprises a nucleic acid sequence that has at least 95% sequence identity to the nucleic acid sequence of SEQ ID NO: 1.

23. The method of claim 22, wherein the vector comprises the nucleic acid sequence of SEQ ID NO: 1.

24. The method of claim 23, wherein at 4 months or more after the initial dose the patient has a supralinear improvement in light sensitivity relative to the improvement with lower doses.

25. The method of claim 24, wherein the visual improvement is measured as one or more of: amplitude of electroretinogram (ERG) response or amplitude or VEP response.

26. The method of any one of claims 20-25, further administering to the patient light stimulation after administering the initial dose.

27. The method of any one of claims 20-25, further administering neurally-coded stimulation after administering the initial dose.

28. The method of any one of claims 1-27, wherein 4 months or more after the initial dose, the patient's light perception threshold has decreased, as measured by a staircase or binary search procedure.

29. The method of any one of claims 1-27, wherein 3 months or more after the initial dose, the patient's ability to detect motion and/or direction of motion has increased as measured by a standard two-alternative forced choice paradigm.

30. The method of any one of claims 1-27, wherein 3 months or more after the initial dose, the patient's shape detection ability has increased as measured by a standard two-alternative forced choice paradigm.

31. The method of any one of claims 1-27, wherein 3 months or more after the initial dose, the patient has an increased ability to detect and/or distinguish colors as measured by a visual acuity test or a standard two-alternative forced choice paradigm.

32. The method of any one of claims 1-31, wherein the retinal degenerative disorder is one or more of: retinitis pigmentosa and macular degeneration.

33. The method of any one of claims 1-32, further comprising administering an oral corticosteroid to the patient 1 to 3 days prior to the intravitreal administration.

34. The method of claim 33, wherein the corticosteroid is oral prednisone/prednisolone administered 1 mg/kg prednisone/prednisolone for 1-3 days prior to injection or on day of injection and optionally continuing at this dose for 7 days after the injection including the injection day a total of 10 days.

35. The method of any one of claims 1-34, further comprising administering valacyclovir 1000 mg/day or acyclovir 400 mg twice a day orally starting 3-7 days prior to the intravitreal administration.

36. A polynucleotide comprising a nucleic acid sequence encoding an optogenetic fusion protein operably linked to a CAG promoter, wherein
the optogenetic fusion protein comprises a light-activated ion channel protein fused to the N-terminus of a reporter protein, and wherein
the nucleic acid sequence encoding the light-activated ion channel protein comprises the nucleic acid sequence of SEQ ID NO: 4.

37. The polynucleotide of claim 36, wherein the polynucleotide comprises DNA.

38. The polynucleotide of claim 36, wherein the polynucleotide comprises RNA.

39. The polynucleotide of claim 36, wherein the reporter protein is GFP.

40. The polynucleotide of claim 39, wherein the optogenetic fusion protein is encoded by a nucleic acid molecule that has a nucleic acid sequence that is at least 90%, 91%, 92%, 93%, 94%, 95%, 96%, 97%, 98%, or 99% identical to the nucleic acid sequence of SEQ ID NO: 2.

41. The polynucleotide of claim 40, wherein the optogenetic fusion is encoded by a nucleic acid molecule that has the nucleic acid sequence of SEQ ID NO: 2.

42. A vector comprising the polynucleotide of any one of claims 1-41, optionally wherein the vector is a plasmid, a DNA vector, an RNA vector, a virion, or a viral vector.

43. The vector of claim 42, wherein the vector is a viral vector.

44. The vector of claim 43, wherein the viral vector is selected from the group comprising an adeno-associated virus (AAV), an adenovirus, a lentivirus, a retrovirus, a poxvirus, a baculovirus, a herpes simplex virus, a vaccina virus, and a synthetic virus.

45. The vector of claim 44, wherein the viral vector is an AAV2.

46. The AAV of claim 44 or 45, wherein the AAV further comprises two inverted terminal repeats (ITRs), wherein the two ITRs comprise a first ITR (ITR1) and a second ITR (ITR2), wherein ITR1 is positioned 5′ to the polynucleotide and ITR2 is position 3′ to the polynucleotide to form a cassette comprising the structure ITR1-optogenetic fusion protein-ITR2.

47. The AAV of claim 46, wherein the two ITRs are AAV serotype 2 ITRs.

48. The vector of any one of claims 42-47, wherein the vector comprises a nucleic acid molecule that is at least 90%, 91%, 92%, 93%, 94%, 95%, 96%, 97%, 98%, or 99% identical to the nucleic acid sequence of SEQ ID NO: 1.

49. The vector of claim 48, wherein the vector has the nucleic acid sequence of SEQ ID NO: 1.

50. A plasmid encoding the viral vector of any one of claims 42-49.

51. A pharmaceutical composition comprising the polynucleotide of any one of claims 1-41, the vector of any one of claims 42-49, or the plasmid of claim 50 and a pharmaceutically acceptable carrier, diluent, or excipient.


SEQUENCE LISTING (9 sequences)

SEQ ID NO: 1 - DNA, 3922 bp, synthetic construct
  source 1..3922: mol_type = other DNA
gctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatggaaacagccgccacaatgacccacgcctttatctcagccgtgcctagcgccgaagccacaattagaggcctgctgagcgccgcagcagtggtgacaccagcagcagacgctcacggagaaacctctaacgccacaacagccggagccgatcacggttgcttcccccacatcaaccacggaaccgagctgcagcacaagatcgcagtgggactccagtggttcaccgtgatcgtggctatcgtgcagctcatcttctacggttggcacagcttcaaggccacaaccggctgggaggaggtctacgtctgcgtgatcgagctcgtcaagtgcttcatcgagctgttccacgaggtcgacagcccagccacagtgtaccagaccaacggaggagccgtgatttggctgcggtacagcatgtggctcctgacttgccccgtgatcctgatccacctgagcaacctgaccggactgcacgaagagtacagcaagcggaccatgaccatcctggtgaccgacatcggcaacatcgtgtgggggatcacagccgcctttacaaagggccccctgaagatcctgttcttcatgatcggcctgttctacggcgtgacttgcttcttccagatcgccaaggtgtatatcgagagctaccacaccctgcccaaaggcgtctgccggaagatttgcaagatcatggcctacgtcttcttctgctcttggctgatgttccccgtgatgttcatcgccggacacgagggactgggcctgatcacaccttacaccagcggaatcggccacctgatcctggatctgatcagcaagaacacttggggcttcctgggccaccacctgagagtgaagatccacgagcacatcctgatccacggcgacatccggaagacaaccaccatcaacgtggccggcgagaacatggagatcgagaccttcgtcgacgaggaggaggagggaggagtggcggcaccggtagtagcagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgca

SEQ ID NO: 2 - DNA, 1510 bp, synthetic construct
  source 1..1510: mol_type = other DNA
atggaaacagccgccacaatgacccacgcctttatctcagccgtgcctagcgccgaagccacaattagaggcctgctgagcgccgcagcagtggtgacaccagcagcagacgctcacggagaaacctctaacgccacaacagccggagccgatcacggttgcttcccccacatcaaccacggaaccgagctgcagcacaagatcgcagtgggactccagtggttcaccgtgatcgtggctatcgtgcagctcatcttctacggttggcacagcttcaaggccacaaccggctgggaggaggtctacgtctgcgtgatcgagctcgtcaagtgcttcatcgagctgttccacgaggtcgacagcccagccacagtgtaccagaccaacggaggagccgtgatttggctgcggtacagcatgtggctcctgacttgccccgtgatcctgatccacctgagcaacctgaccggactgcacgaagagtacagcaagcggaccatgaccatcctggtgaccgacatcggcaacatcgtgtgggggatcacagccgcctttacaaagggccccctgaagatcctgttcttcatgatcggcctgttctacggcgtgacttgcttcttccagatcgccaaggtgtatatcgagagctaccacaccctgcccaaaggcgtctgccggaagatttgcaagatcatggcctacgtcttcttctgctcttggctgatgttccccgtgatgttcatcgccggacacgagggactgggcctgatcacaccttacaccagcggaatcggccacctgatcctggatctgatcagcaagaacacttggggcttcctgggccaccacctgagagtgaagatccacgagcacatcctgatccacggcgacatccggaagacaaccaccatcaacgtggccggcgagaacatggagatcgagaccttcgtcgacgaggaggaggagggaggagtggcggcaccggtagtagcagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgagg

SEQ ID NO: 3 - protein, 569 aa, synthetic construct
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGVAAPVVAVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 4 - DNA, 975 bp, synthetic construct
  source 1..975: mol_type = other DNA
atggaaacagccgccacaatgacccacgcctttatctcagccgtgcctagcgccgaagccacaattagaggcctgctgagcgccgcagcagtggtgacaccagcagcagacgctcacggagaaacctctaacgccacaacagccggagccgatcacggttgcttcccccacatcaaccacggaaccgagctgcagcacaagatcgcagtgggactccagtggttcaccgtgatcgtggctatcgtgcagctcatcttctacggttggcacagcttcaaggccacaaccggctgggaggaggtctacgtctgcgtgatcgagctcgtcaagtgcttcatcgagctgttccacgaggtcgacagcccagccacagtgtaccagaccaacggaggagccgtgatttggctgcggtacagcatgtggctcctgacttgccccgtgatcctgatccacctgagcaacctgaccggactgcacgaagagtacagcaagcggaccatgaccatcctggtgaccgacatcggcaacatcgtgtgggggatcacagccgcctttacaaagggccccctgaagatcctgttcttcatgatcggcctgttctacggcgtgacttgcttcttccagatcgccaaggtgtatatcgagagctaccacaccctgcccaaaggcgtctgccggaagatttgcaagatcatggcctacgtcttcttctgctcttggctgatgttccccgtgatgttcatcgccggacacgagggactgggcctgatcacaccttacaccagcggaatcggccacctgatcctggatctgatcagcaagaacacttggggcttcctgggccaccacctgagagtgaagatccacgagcacatcctgatccacggcgacatccggaagacaaccaccatcaacgtggccggcgagaacatggagatcgagaccttcgtcgacgaggaggaggagggaggagtg

SEQ ID NO: 5 - protein, 325 aa, synthetic construct
METAATMTHAFISAVPSAEATIRGLLSAAAVVTPAADAHGETSNATTAGADHGCFPHINHGTELQHKIAVGLQWFTVIVAIVQLIFYGWHSFKATTGWEEVYVCVIELVKCFIELFHEVDSPATVYQTNGGAVIWLRYSMWLLTCPVILIHLSNLTGLHEEYSKRTMTILVTDIGNIVWGITAAFTKGPLKILFFMIGLFYGVTCFFQIAKVYIESYHTLPKGVCRKICKIMAYVFFCSWLMFPVMFIAGHEGLGLITPYTSGIGHLILDLISKNTWGFLGHHLRVKIHEHILIHGDIRKTTTINVAGENMEIETFVDEEEEGGV

SEQ ID NO: 6 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 7 - DNA, 588 bp, synthetic construct
  source 1..588: mol_type = other DNA
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 8 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 9 - DNA, 3656 bp, synthetic construct
  source 1..3656: mol_type = other DNA
tgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccgccaccatggaaacagccgccacaatgacccacgcctttatctcagccgtgcctagcgccgaagccacaattagaggcctgctgagcgccgcagcagtggtgacaccagcagcagacgctcacggagaaacctctaacgccacaacagccggagccgatcacggttgcttcccccacatcaaccacggaaccgagctgcagcacaagatcgcagtgggactccagtggttcaccgtgatcgtggctatcgtgcagctcatcttctacggttggcacagcttcaaggccacaaccggctgggaggaggtctacgtctgcgtgatcgagctcgtcaagtgcttcatcgagctgttccacgaggtcgacagcccagccacagtgtaccagaccaacggaggagccgtgatttggctgcggtacagcatgtggctcctgacttgccccgtgatcctgatccacctgagcaacctgaccggactgcacgaagagtacagcaagcggaccatgaccatcctggtgaccgacatcggcaacatcgtgtgggggatcacagccgcctttacaaagggccccctgaagatcctgttcttcatgatcggcctgttctacggcgtgacttgcttcttccagatcgccaaggtgtatatcgagagctaccacaccctgcccaaaggcgtctgccggaagatttgcaagatcatggcctacgtcttcttctgctcttggctgatgttccccgtgatgttcatcgccggacacgagggactgggcctgatcacaccttacaccagcggaatcggccacctgatcctggatctgatcagcaagaacacttggggcttcctgggccaccacctgagagtgaagatccacgagcacatcctgatccacggcgacatccggaagacaaccaccatcaacgtggccggcgagaacatggagatcgagaccttcgtcgacgaggaggaggagggaggagtggcggcaccggtagtagcagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatttgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactaca